(19) United States
(12) Patent Application Publication
(10) Pub. No.: US 2023/0301568 A1
(43) Pub. Date: Sep. 28, 2023

(54) CAPILLARY BLOOD SAMPLING DEVICE AND METHOD OF USING THE SAME

(71) Applicant: Preci Health SA, Neuchâtel (CH)

(72) Inventors: Alain JACCARD, Ste-Croix (CH); Lucien VOUILLAMOZ, Feusisberg (CH); Philippe MARGAIRAZ, La Chaux-de-Fonds (CH); Robert Sean HÄGEN, Ozona, FL (US); Gary GOGOLIN, Tampa, FL (US); Rolando Abaroa MARTINEZ, Greifensee (CH); Shy ZIMANN, Pardes Hanna Karkur (IL)

(73) Assignee: Preci Health SA, Neuchâtel (CH)

(21) Appl. No.: 18/022,772

(22) PCT Filed: Aug. 23, 2021
(86) PCT No.: PCT/IB2021/000580

§ 371 Date: Feb. 23, 2023

Related U.S. Application Data
(60) Provisional application No. 63/069,112, filed on Aug. 23, 2020.
(60) Provisional application No. 63/142,756, filed on Jan. 28, 2021.
(60) Provisional application No. 63/150,113, filed on Feb. 17, 2021.
(60) Provisional application No. 63/153,088, filed on Feb. 24, 2021.

(30) Foreign Application Priority Data
Mar. 31, 2021 (WO) PCTIB2021/000187

(51) Int. Cl. A61B 5/151; A61B 5/15

(52) U.S. Cl. CPC A61B 5/151; A61B 5/150022; A61B 5/150748; A61B 5/150099; A61B 5/150442

(57) ABSTRACT

A body fluid sampling device (100) provides a user with the ability to sample and/or analyze a fluid in the body (body fluid) such as blood, puss or venom. The device (100) includes at least: a body fluid reservoir for containing the sampled body fluid and a fluid extraction mechanism including at least one conduit or needle connected to a conduit injection and retraction mechanism and a vacuum reservoir. After connecting the body fluid reservoir to the sampling device, a vacuum seal of the body fluid reservoir is adapted to be broken so as to cause the sucking of the body fluid into the body fluid reservoir. The sampling device (100) aids in executing a method (200) of the invention consisting of collecting body fluid such as capillary blood samples without the intervention of medically trained personnel. The sampling device (100) enables the user to: (a) sample a body fluid, optionally auto-sampling; (b) optionally, using one or more droplet(s) of the sampled body fluid, to immediately analyze the body fluid; and (c) provide a medical analysis tube (4000) meeting size and interface standards filled with the sampled body fluid for analysis in a point of care or medical lab. The sampling device includes a vacuum tube and an interface therefor. The vacuum tube provides the suction necessary to draw the body fluid from the user/patient and to fill the vacuum tube with the body fluid.

### CROSS REFERENCE TO RELATED APPLICATIONS

This application claims the benefit of U.S. Provisional Applications No. 63/069,112, filed Aug. 23, 2020, entitled CAPILLARY BLOOD SAMPLING DEVICE AND METHOD OF USING THE SAME, No. 63/142,756, filed Jan. 28, 2021, entitled CAPILLARY BLOOD SAMPLING SYSTEM INCLUDING USER/PATIENT AUTHENTICATION, No. 63/153,088, filed Feb. 24, 2021, entitled VACCINE AND/OR DRUG INJECTION AND ADMINISTRATION DEVICE, No. 63/150,113, filed Feb. 17, 2021, entitled CAPILLARY BLOOD SAMPLING DEVICE, and International Patent Application No. PCT/IB2021/000187, filed Mar. 31, 2021, entitled BODY FLUIDS SAMPLING DEVICE AND METHOD OF USING THE SAME, the content of the entirety of which is explicitly incorporated herein by reference and relied upon to define features for which protection may be sought hereby as it is believed that the entirety thereof contributes to solving the technical problem underlying the invention, some features that may be mentioned hereunder being of particular importance.

### COPYRIGHT & LEGAL NOTICE

A portion of the disclosure of this patent document contains material which is subject to copyright protection. The Applicant has no objection to the facsimile reproduction by anyone of the patent document or the patent disclosure as it appears in the Patent and Trademark Office patent file or records, but otherwise reserves all copyright rights whatsoever. Further, no references to third party patents or articles made herein is to be construed as an admission that the present invention is not entitled to antedate such material by virtue of prior invention.

### BACKGROUND OF THE INVENTION

This invention relates to devices allowing for self-sampling of body fluids without the intervention of medical personnel. In cases of war, large epidemics or pandemics, it may become necessary to analyze body fluids, for example blood, of a substantial part of a given population, potentially the whole population of a neighborhood, suburb, a city, a whole country, a continent or even the entire planet. In such situation, it may be impossible for medical personnel to take a sampling of the body fluids of every patient, due to confinement, risk of contamination, unsafe areas, travel distances, lack of transport infrastructures, or lack of personnel.

What is needed is a fluid sampling device adapted to be used safely by any individual on themselves without any medical training and able to provide the necessary fluid samples necessary for home analysis devices or analysis systems at the point of care or at a medical laboratory.

### SUMMARY OF THE INVENTION

A disposable body fluid sampling device provides a user with the ability to sample and/or analyze a fluid in the body (body fluid) such as blood, puss or venom. The device includes at least: a body fluid reservoir for containing the sampled body fluid and a fluid extraction mechanism including at least one conduit or needle connected to a vacuum reservoir. When connecting the vacuum reservoir to the body fluid reservoir, a vacuum seal of the vacuum reservoir is adapted to be broken so as to cause the sucking of the body fluid into the body fluid reservoir. The sampling device aids in executing a method of the invention consisting of collecting body fluid such as capillary blood samples without the intervention of medically trained personnel. The sampling device enables the user to: (a) sample a body fluid, optionally auto-sampling; (b) optionally, using one or more droplet(s) of the sampled body fluid, to immediately analyze the body fluid; and (c) provide a medical analysis tube meeting size and interface standards filled with the sampled body fluid for analysis in a point of care or medical lab. The sampling device includes a vacuum tube and an interface therefor. The vacuum tube provides the suction necessary to draw the body fluid from the user/patient and to fill the vacuum tube with the body fluid.

### BRIEF DESCRIPTION OF THE DRAWINGS

FIG. 1 is a side view of a first embodiment of the capillary blood sampler device of the invention.

FIG. 2 is a partial cross-sectional side view of the embodiment of FIG. 1.

FIG. 3A is a cross-sectional side view of the embodiment of FIG. 1 at the step 1 of the method.

FIG. 3B is a cross-sectional side view of the embodiment of FIG. 1 at the step 2 of the method.

FIG. 3C is a cross-sectional side view of the embodiment of FIG. 1 at the step 3 of the method.

FIG. 3D is a cross-sectional side view of the embodiment of FIG. 1 at the step 4 of the method.

FIG. 3E is a cross-sectional side view of the embodiment of FIG. 1 at the step 5 of the method.

FIG. 3F is a cross-sectional side view of the embodiment of FIG. 1 at the step 6 of the method.

FIG. 3G is a cross-sectional side view of the embodiment of FIG. 1 at the step 7 of the method.

FIG. 3H is a cross-sectional side view of the embodiment of FIG. 1 at the step 8 of the method.

FIG. 3I is a cross-sectional side view of the embodiment of FIG. 1 at the step 9 of the method.

FIG. 4A is a side view, before use, of a first embodiment of the cutting blade used in the device of the invention.

FIG. 4B is a side view of a first embodiment of the cutting blade used in the device of the invention while in use.

FIG. 4C is a side view of a first embodiment of the cutting blade used in the device of the invention after use.

FIG. 5A is a general view of a second embodiment of the cutting blade used in the device of the invention before use.

FIG. 5B is a side view of the cutting blade of FIG. 5A before use.

FIG. 5C is a front view of the cutting blade of FIG. 5A before use.

FIG. 5D is a top view of the cutting blade of FIG. 5A before use.

FIG. 5E is a side view of the cutting blade of FIG. 5A while in use.

FIG. 5F is a side view of the cutting blade of FIG. 5A after use.

FIG. 6A is a side view of a third embodiment of the cutting blade used in the device of the invention before use.

FIG. 6B is a side view of the cutting blade of FIG. 6A after use.

FIG. 7A is a cross-sectional side view of the embodiment of FIG. 1, comprises an anticoagulant substance.

FIG. 7B is a cross-sectional side view of the embodiment of FIG. 7A in contact with the user/patient through its suction interface.

FIG. 8 is a cross-sectional side view of a variant of the embodiment of FIG. 1 in contact with the user/patient through its suction interface, wherein the volume of the suction chamber 1400 is reduced.

FIGS. 9A and 9B are cross-sectional side views of a variant of the embodiment of FIG. 2, wherein the embodiment comprises an elastic and within a cavity movable membrane, and the embodiment as shown is in contact with the user/patient through its suction interface.

FIGS. 10A and 10B are cross-sectional side views of still another variant of the embodiment of FIG. 2, wherein the embodiment comprises an elastic and within a cavity movable membrane, and the embodiment as shown is in contact with the user/patient through its suction interface.

FIG. 11 shows an alternative method of body fluid sampling, using the second and third embodiments of the device of the invention.

FIG. 12A to FIG. 12C shows an example of a mechanism of a lacerating device that can be used with the second and a third embodiments of the device of the invention.

FIG. 13A is an upper, perspective view of the first and second embodiments of the blood sampling device of the invention.

FIG. 13B is a lower, perspective view of the first and second embodiments of the blood sampling device of the invention.

FIG. 14A is an upper, perspective view of the third embodiment of the body fluid sampling device of the invention before use.

FIG. 14B is a lower, perspective view of the third embodiment of the body fluid sampling device of the invention before use.

FIG. 14C is a top view of the third embodiment of the body fluid sampling device of the invention before use.

FIG. 14D is a bottom view of the third embodiment of the body fluid sampling device of the invention before use.

FIG. 14E is a left side view of the third embodiment of the body fluid sampling device of the invention before use.

FIG. 14F is a right side view of the third embodiment of the body fluid sampling device of the invention before use.

FIG. 14G is a front side view of the third embodiment of the body fluid sampling device of the invention before use.

FIG. 14H is a back side view of the third embodiment of the body fluid sampling device of the invention before use.

FIG. 14I is an upper, perspective view of the third embodiment of the body fluid sampling device of the invention while in use.

FIG. 14J is a lower, perspective view of the third embodiment of the body fluid sampling device of the invention while in use.

FIG. 14K is a top view of the third embodiment of the body fluid sampling device of the invention while in use.

FIG. 14L is a bottom view of the third embodiment of the body fluid sampling device of the invention while in use.

FIG. 14M is a left side view of the third embodiment of the body fluid sampling device of the invention while in use.

FIG. 14N is a right side view of the third embodiment of the body fluid sampling device of the invention while in use.

FIG. 14O is a front side view of the third embodiment of the body fluid sampling device of the invention while in use.

FIG. 14P is a back side view of the third embodiment of the body fluid sampling device of the invention while in use.

FIG. 15A shows an adhesive pad of the invention before use.

FIG. 15B shows an adhesive pad of the invention while in use.

FIG. 16A illustrates the step of installing the adhesive pad of the invention.

FIG. 16B illustrates the step of installing the fluid sampling device of the invention.

FIG. 16C illustrates the step of activating the sampling with the device of the invention.

FIG. 16D illustrates the step of waiting and monitoring the sampling with the device of the invention.

FIG. 16E illustrates the step of removing the device of the invention after sampling.

FIG. 16F illustrates the step of dressing the wound with the adhesive pad of the invention.

FIG. 17 shows a cross section lateral view of the suction chamber of the fluid sampling device of the invention.

FIGS. 18A to 18P are schematic views illustrating the steps of a fourth embodiment of the method of using the system of the invention.

FIGS. 19A to 19D are schematic views with more detail about the specific features of the adhesive integrated dressing included in the capillary blood sampling device.

FIG. 20A is a perspective view of a fourth embodiment of the cutting blade of a device of the invention is made of a single part.

FIG. 20B is a side, four-position, shutter view of the fourth embodiment of the cutting blade of a device of the invention.

FIG. 20C is a side, four-position, shutter view of the fourth embodiment of the cutting blade of a device of the invention showing the retraction thereof.

FIG. 21A is a perspective view of a fifth embodiment of the cutting blade of the invention.

FIG. 21B is a front view of the fifth embodiment of the cutting blade of the invention.

FIG. 22A is a side view of a sixth embodiment the cutting blade of a device of the invention, prior to entry into the epidermal layer of the patient's body.

FIG. 22B is a side view of the sixth embodiment the cutting blade of a device of the invention half way through its cycle of motion.

FIG. 22C is a side view of the sixth embodiment the cutting blade of a device of the invention at the end of its cycle of motion, having returned to its initial position.

FIG. 23A is a perspective view of a seventh embodiment the cutting blade of a device of the invention showing a standard scalpel blade.

FIG. 23B is a side view of the seventh embodiment the cutting blade of a device of the invention, prior to entry into the epidermal layer.

FIG. 23C is a side view of the seventh embodiment of the cutting blade of a device of the invention after entry into the epidermal layer, as it is about to be removed from said layer.

FIG. 23D is a side view of the epidermal layer showing the cut made by the seventh embodiment of the cutting blade.

FIG. 24A to FIG. 24C show examples of visible features that can be integrated in a device of the invention to be easily recognized by the human observer, or easily identified by a real-time image analysis software.

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended for distinguishing between similar elements and not necessarily for describing a sequential or chronological order. Moreover, relative terms like ‘front’, ‘back’, ‘top’ and ‘bottom’, and the like in the Description and/or in the claims are not necessarily used for describing exclusive relative position. Those skilled in the art will therefore understand that such terms may be interchangeable with other terms, and that the embodiments described herein are capable of operating in other orientations than those explicitly illustrated or otherwise described.

### DETAILED DESCRIPTION OF THE PREFERRED EMBODIMENT

The following description is not intended to limit the scope of the invention in any way as they are exemplary in nature, serving to describe the best mode of the invention known the inventors as of the filing date hereof. Consequently, changes may be made in the arrangement and/or function of any of the elements described in the exemplary embodiments disclosed herein without departing from the spirit and scope of the invention.

The capillary blood sampling device according the invention may take different sizes and shapes, depending on the size of the sample to be collected, and on the area of the body where the sample is to be collected. A reactant may be added by the user on the surface of the skin to facilitate the conservation of the sample or to enable specific analyses. The capillary blood sampling device may be equipped with a unique identification code, and may carry an electronically readable identification tag. Depending on the circumstances the sampling device may be equipped with geolocalization (GPS) and long-range communication capabilities so as to be collectable without any further action from the user after the sampling process has been executed.

The capillary blood sampling device according the invention is made to use a medical analysis vacuum tube meeting size and interface standards as well-known in the industry and commonly used for venous blood collection, so that the tubes' content can be analyzed on standard automatized analysis equipment. These tubes are available in standard form with various levels of vacuum. Nevertheless, for some embodiments of the invention, larger tubes and/or higher vacuums may be advantageous.

The sampling device 100 aids in executing a method 200 of the invention consisting of collecting body fluid such as capillary blood samples without the intervention of medically trained personnel. The sampling device 100 enables the user to: (a) sample a body fluid, optionally auto-sampling; (b) optionally, using one or more droplet(s) of the sampled body fluid, to immediately analyze the body fluid; and (c) provide a medical analysis tube (4000) meeting size and interface standards filled with the sampled body fluid for analysis in a point of care or medical lab. The sampling device includes a vacuum tube and an interface therefor. The vacuum tube provides the suction necessary to draw the body fluid from the user/patient and to fill the vacuum tube with the body fluid. The vacuum tube may be a standard medical analysis tube (4000) or one that is customized but meets certain standard as to size or interface nonetheless.

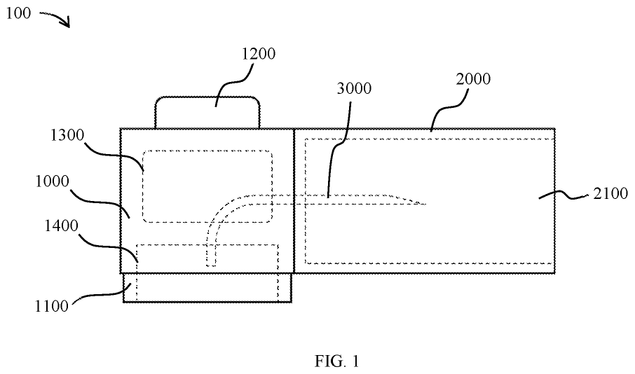

Referring now to FIG. 1, an embodiment of capillary blood sampling device 100 according the invention has a main structure 1000 containing the suction interface 1100 adapted to be attached to the skin of the user/patient, one or more push-button(s) 1200 for the user/patient to actuate the device 100, and a tube holder 2000 destined to receive vacuum sampling tubes similar to the vacuum sampling tubes well-known in the industry for venous blood sampling. Inside of the main structure 1000 are located a suction chamber 1400, connected to the inside 2100 of the tube holder 2000 via a channel 3000, a mechanism 1300 to convert the action of the user/patient pushing on the one or more buttons 1200 into actuation of the device 100, and optional electronic and/or connectivity features such as GPS or geolocalization system, identification tag, wireless communication system, or countdown with audible feedback, etc.

The device's structure 1000 preferably includes two or more push buttons 1200 and the mechanism 1300 ensures that only the activation of all push-buttons 1200 launches the sampling process, so that the risk of inadvertent launching is minimized. The mechanism 1300 may include a combination of levers, stoppers, cams, or any other mechanical elements well known in the industry, or may preferably be made of flexible elements that can release the sampling just by being deformed when the user/patient presses the push-button(s) 1200. The device's structure 1000 may also include means to expel a small quantity of the blood from the device 100 for quick on-site analysis.

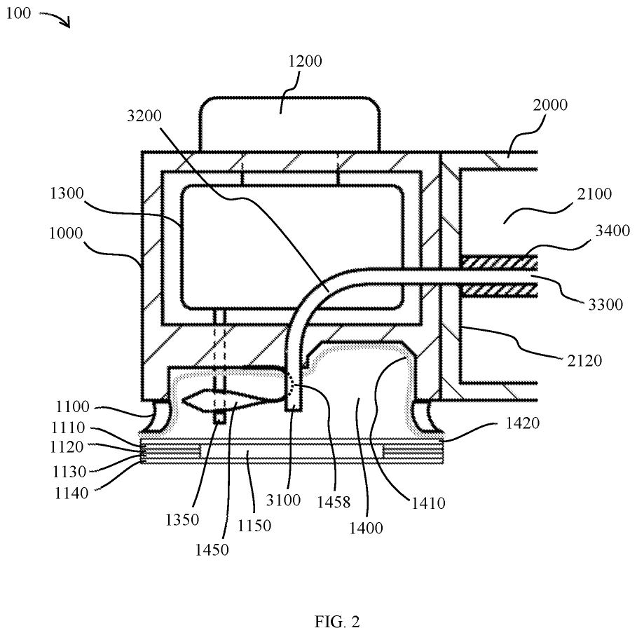

Referring now to FIG. 2, an embodiment of capillary blood sampling device 100 according the invention is in contact with the user/patient through its suction interface 1100, the suction interface 1100 attached to a bandage 1120 with permanent glue 1110, the bandage 1120 having on its surface facing the user/patient's skin a temporary glue 1130 adapted to hold the device 100 unto the user/patient's skin at least for the duration of the sampling process. The temporary glue 1130 is protected before device's use by a removable protective film 1140. The glues 1110, 1130 and bandage 1120 present a passage 1150 to allow the blood sampling, and are air-tight on their periphery in order to ensure the build-up of vacuum between the user/patient's skin and the suction chamber 1400 for the sampling process. The bandage 1120 may have a relatively large surface compared to the surface of the suction interface, in order to ensure sufficient air-tightness with the user/patient's skin.

The suction chamber 1400 contains 1 or more lancet(s) 1450, in principle 1 for up to 500 mul, and 2 if 1 ml need be collected, as required depending on the sample size to be collected. For the reader's information, the duration of blood flow is about 30′ for 500 mul for one incision, and so about the same time for 1 ml using two incisions. The lancet 1450 has an elastic or spring portion 1458 which has been loaded at the assembly of the device 100, and is held under tension by a mechanical finger 1350. The mechanical finger 1350 is linked to the mechanism 1300 so that the mechanism 1300 can release the lancet 1450 when actuated. The lancet is positioned so as to lacerate the user/patient' skin through the passage 1150 when released. The suction chamber is closed by an air-tight membrane 1420 made of an air-tight material that can be lacerated by the lancet 1450 while at the same time cutting the user/patient's skin without tearing. Optionally, the suction chamber 1400 contains an air-tight elastic lining 1410 which allows the mechanism 1300 to actuate the mechanical finger 1350 in an air-tight manner, and optionally includes means to expel a small quantity of blood for quick on-site analyses. Appropriate materials for the membrane and the optional elastic lining 1410 are well-known in the industry and may include silicone, rubber, and other elastomers and/or plastics in one or more layers. The suction chamber 1400 is made so as to minimize its volume, so that the majority of the collected blood doesn't remain in the suction chamber 1400 and can be fed into the vacuum tube, and a minimal part of the vacuum provided by the tube is used to establish vacuum in the suction chamber 1400. The channel 3000 connecting the suction chamber 1400 to the inside 2100 of the tube holder 2000 may be a conduit or needle 3200 with the suction end 3100 of the conduit connected to the suction chamber 1400 and the dispensing end 3300 of the conduit adapted to enter the vacuum tube and bring the collected blood into the tube. Typically, the conduit or needle 3200 may be made of stainless steel, but other materials available nowadays in the industry such as other metals, composites and/or plastics may be used.

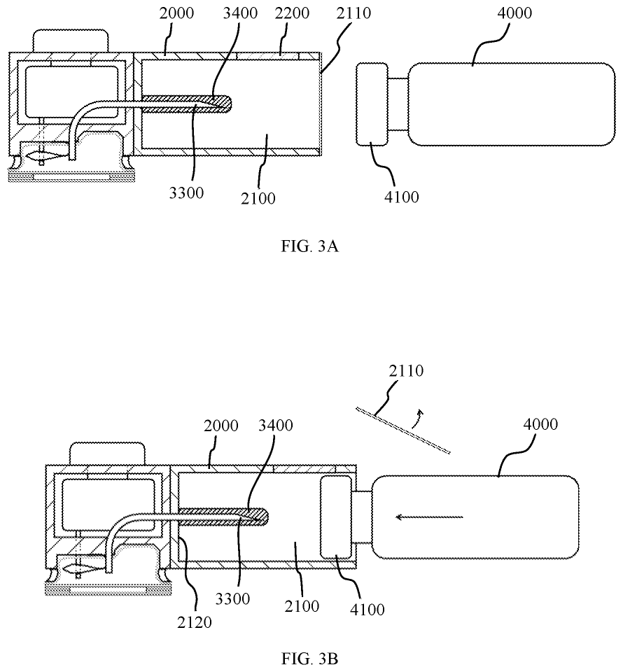

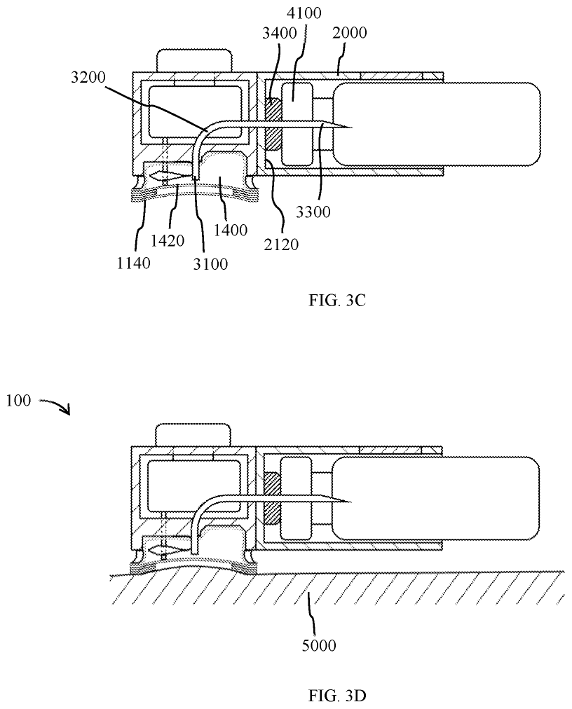

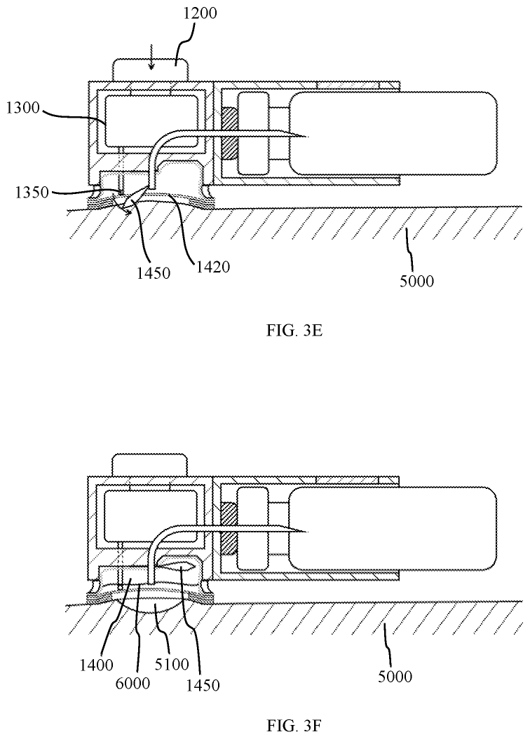

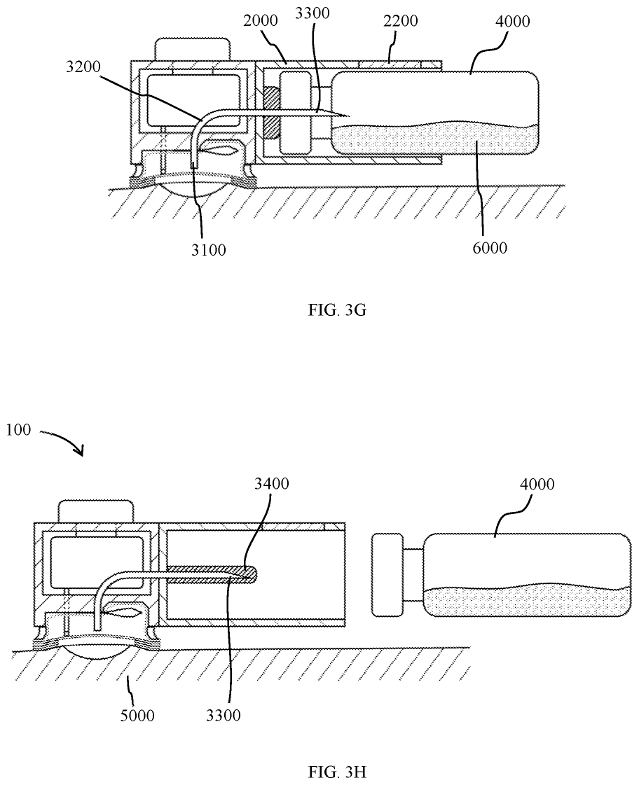

Referring now to FIGS. 3A to 3I, the functioning of the device 100 and a corresponding method 200 can be described as follows:
1. FIG. 3A: The sampling device 100 and the vacuum tube 4000 are delivered separately to the user/patient in sterile packaging. The dispensing end 3300 of the conduit is sharp so as to be able to pierce the septum 4100 (also sometimes called “rubber stopper”) of the vacuum tube 4000, and is protected by an elastic (preferably silicon or rubber) sleeve 3400. Optionally, the tube holder 2000 is closed by a removable protective film 2110. Optionally, the tube holder 2000 is made of transparent material. Alternatively, optionally, the tube holder 2000 contains a transparent window 2200 for the user/patient to see the inside 2100 of the tube holder 2000. Optionally, the rubber sleeve is made of a “self-healing” material such as well-known in the industry so that it automatically closes the dispensing end of the conduit or needle 3300 after use.
2. FIG. 3B: The user/patient removes the optional protective film 2110, and inserts the vacuum tube 4000 in the tube holder 2000, the septum 4100 of the vacuum tube 4000 facing the dispensing end 3300 of the conduit or needle, until reaching the bottom 2120 of the inside 2100 of the tube holder 2000.
3. FIG. 3C: When reaching the bottom 2120 of the inside 2100 of the tube holder 2000, the vacuum tube 4000 compresses the rubber sleeve 3400, stripping the dispensing end of the conduit or needle 3300, allowing the dispensing end of the conduit 3300 to pierce the septum 4100 of the vacuum tube 4000 and establishing an airtight connection from the vacuum tube 4000 to the suction chamber 1400 via the conduit or needle 3200. As a result, the suction chamber 1400 is placed under vacuum.
4. FIG. 3D: The user/patient disinfects the area of skin 5000 where the blood collection is to be made, removes the protective film 1140 and applies the device 100 on the area where the blood collection is to be made. As a result, the device holds and seals against the user/patient's skin 5000 thanks to the bandage 1120 and the glue 1110 and 1130.
5. FIG. 3E: The user/patient actuates the mechanism 1300 by pushing the one or more push-button(s) 1200. The mechanism then actuates the mechanical finger 1350 and releases the one or more lancet(s) 1450. The one or more lancet(s) 1450 cut(s) through the membrane 1420 and the user/patient's skin 5000, lacerating through several capillaries in the patient's skin 5000, and allowing the vacuum to access the user/patient's skin 5000.
6. FIG. 3F: After having lacerated the membrane 1420 and the user/patient's skin 5000, the one or more lancet(s) 1450 terminate(s) its/their movement in a recessed area of the suction chamber 1400 out of the wound area 5100, its sharp edges out of the reach of the user/patient. As the wound 5100 starts bleeding, the suction chamber 1400 is progressively filled with the user/patient's blood 6000.
7. FIG. 3G: The blood 6000 fills the vacuum tube 4000 through the suction end 3100 of the conduit or needle 3200, through the conduit 3200, through the dispensing end of the conduit 3300.
8. FIG. 3H: When the vacuum tube 4000 is sufficiently filled with blood 6000, the user/patient removes the vacuum tube 4000 from the device. Indication that the vacuum tube is sufficiently filled may be provided through an electronic timer integrated in the mechanism 1300, through a graduation on the vacuum tube which the user/patient can see through the transparent tube holder 2000, through a magnetic or capacitance—sensitive strip that is in contact with the blood, or through the transparent window 2200, or by the observation of the stopping of the blood flow when the vacuum has been exhausted, or by any other appropriate means. As the vacuum tube is removed from the tube holder 2000, the elastic sleeve 3400 is free to extend and cover the dispensing end of the conduit or needle 3300, closing the path for the blood 6000. Optionally, the user/patient inserts a further vacuum tube to collect a further blood sample, repeating steps 7 and 8.
9. FIG. 3I: The user/patient removes the device 100 and applies a typically separately purchased, small-wounds bandage 5200 on the wound 5100. The blood remaining in the suction chamber 1400 is retained in the suction chamber 1400 by the membrane 1420. In case of need for a quick on-site analysis, a few drops of the blood contained in the suction chamber 1400 can be obtained by pressing on the membrane 1420. Optionally a second mechanism (not represented) integrated in the structure 1000 provides the means to expel a small quantity of blood by squeezing the elastic lining upon pressing a push-button.
10. User sends or brings the tube 4000 to a medical analyses lab or to a point of care to be analyzed. The user/patient's own refrigerator and, optionally, a container with high thermal inertia such as that described in US application no. U.S. 63/002,581, or U.S. 63/006,337, the contents of which are incorporated herein by reference and relied upon, or a container with high thermal inertia and/or thermal insulation optionally equipped with temperature monitoring and/or signalling, may also be used to facilitate the delivery of a suitable sample to a laboratory.
11. The analysis lab analyzes the blood sample and communicates the test results to the patient and/or to the relevant authorities.
12. Optionally treatment and/or quarantine protocol is initiated to ensure that a test subject having a positive test is handled in a manner to minimize the spread of the pathogen.

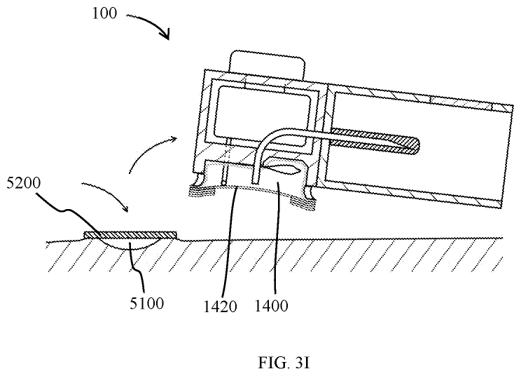

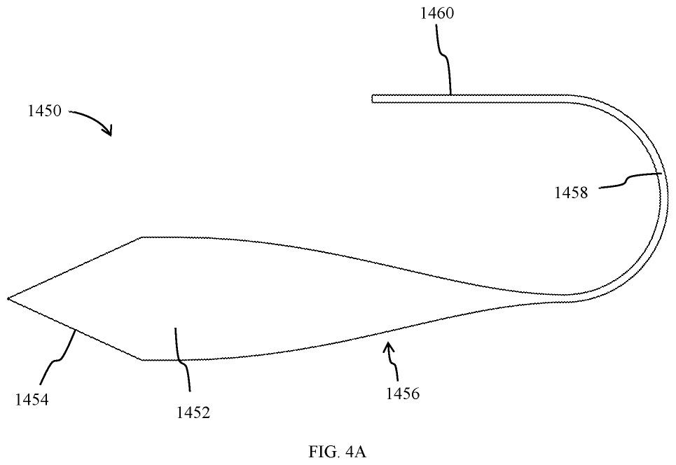

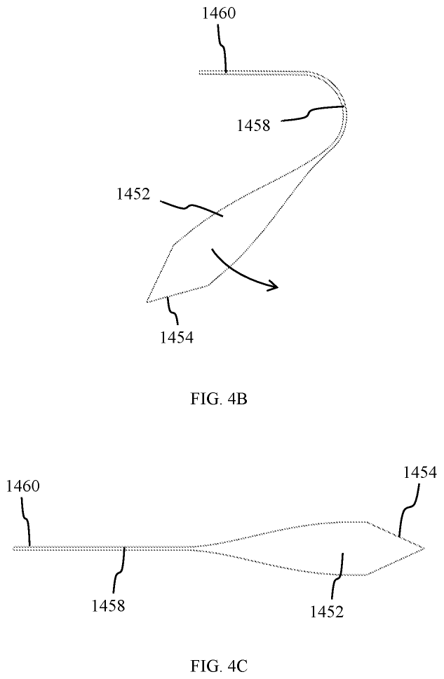

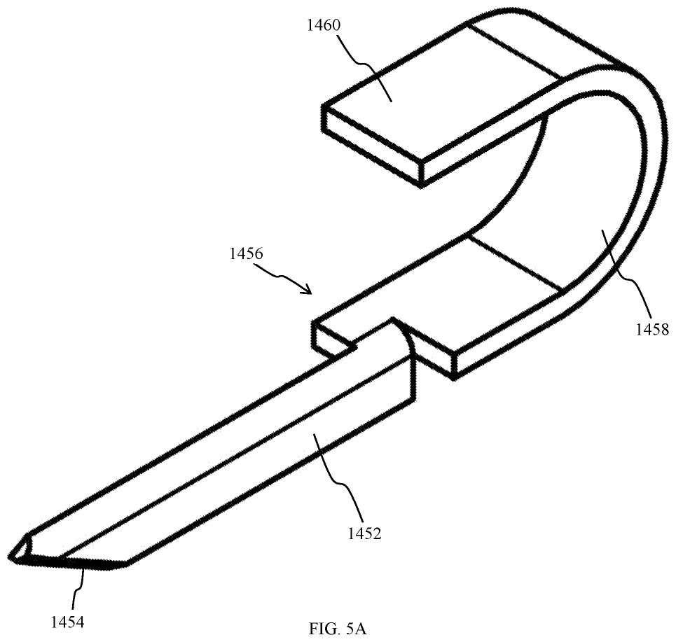

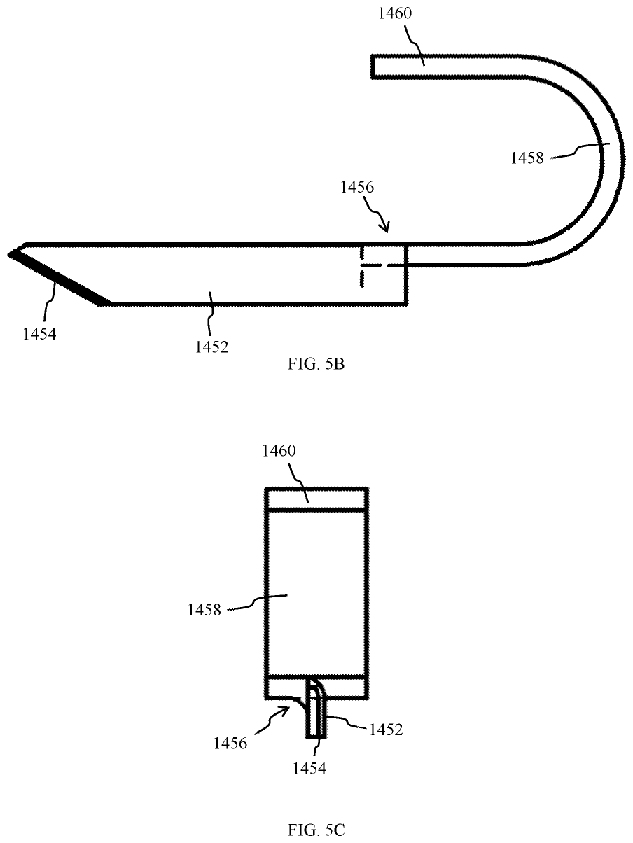

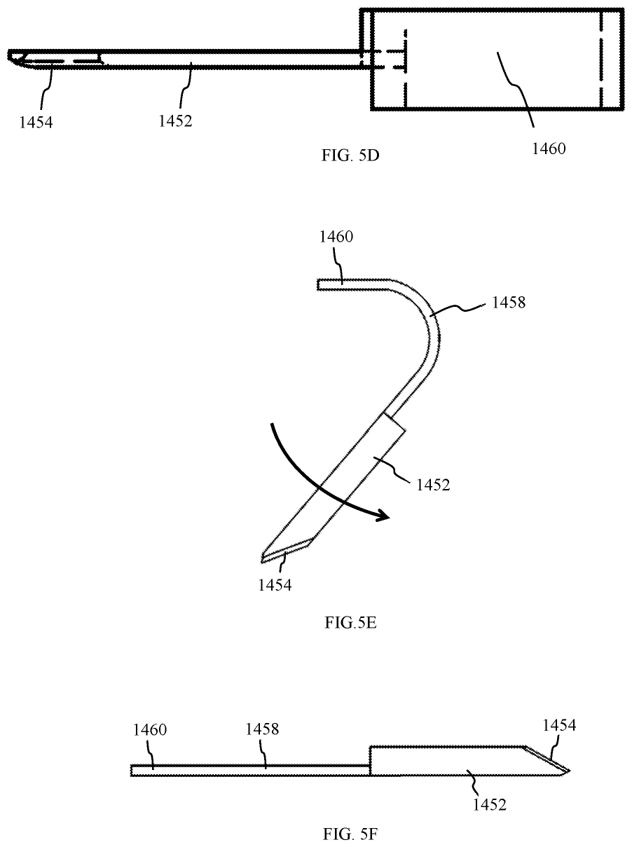

Referring now to FIGS. 4A to 4C, FIGS. 5A to 5F and FIGS. 6A to 6B showing three different embodiments of the lancet(s) 1450, the lancet(s) 1450 is made of a single part, typically out of sheet metal, but may be also made of other materials, including composite materials presenting the appropriate mechanical properties. The lancet has an end 1460 facing the sampling device structure 1000 so that it can be attached to it, followed by an elastic zone 1458 which is bent elastically when the lancet is ready to be used. In this way, the lancet 1450 contains all the energy necessary for its movement. The elastic zone 1458 has an appropriate, preferably flat cross-section so as to provide a preferred release trajectory perpendicular to its attachment end 1460. The release trajectory is illustrated by a bent arrow in FIG. 4B and FIG. 5E. After the elastic zone 1458, the lancet is twisted by 90° in the area 1456 so as to provide a blade 1452 which is in the same plane as the release trajectory. The blade 1452 has a high rigidity in the direction of the release trajectory. Towards the end of the blade 1452, a cutting edge 1454 is provided so as the lacerate the membrane 1420 and the user/patient's skin 5000. After completion of the movement of the lancet 1450, the cutting edge 1454 faces the same side as the attachment end 1460, away from the user/patient's reach.

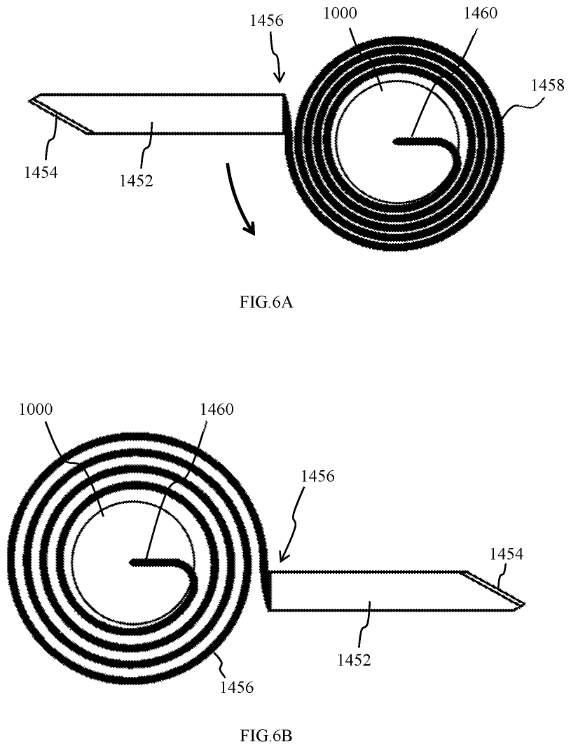

Referring now in particular to FIGS. 6A and 6B, in the third embodiment the elastic zone is bent over several turns, taking the shape of a spiral spring as well known in horology mechanisms.

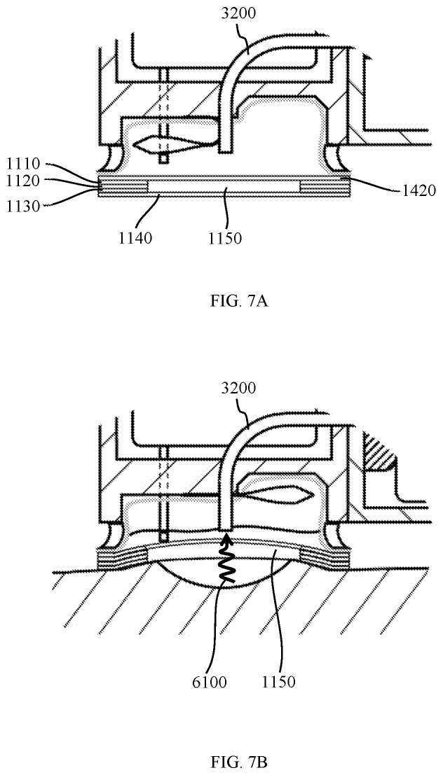

Referring now to FIGS. 7A and 7B, the passage 1150 is optionally filled with an anticoagulant substance, such as Heparin. The sampled blood 6100 (see FIG. 7B) travels through this anticoagulant substance and mixes with it, so that the coagulation of the sampled blood 6100 is slowed down and clogging of the conduit or needle 3200 is prevented.

Referring now to FIG. 8, the volume of the suction chamber 1400 has to be kept as small as possible, with smooth surface transitions to avoid favoring the local coagulation of the sampled blood 6100. Particular attention must be exercised in ensuring a smooth surface transition at the location of the connection between the suction chamber 1400 and the conduit or needle 3200. Optionally, the internal surface of the suction chamber may be coated with an anticoagulant substance 1430, such as Heparin. The sampled blood 6100 mixes with this anticoagulant substance in the suction chamber 1400, so that the coagulation of the sampled blood 6100 is slowed down and clogging of the conduit or needle 3200 is prevented.

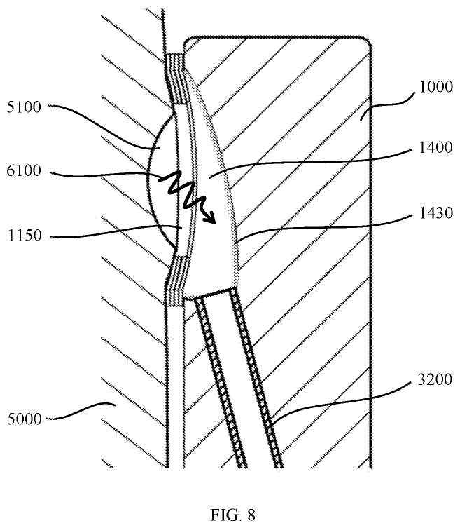

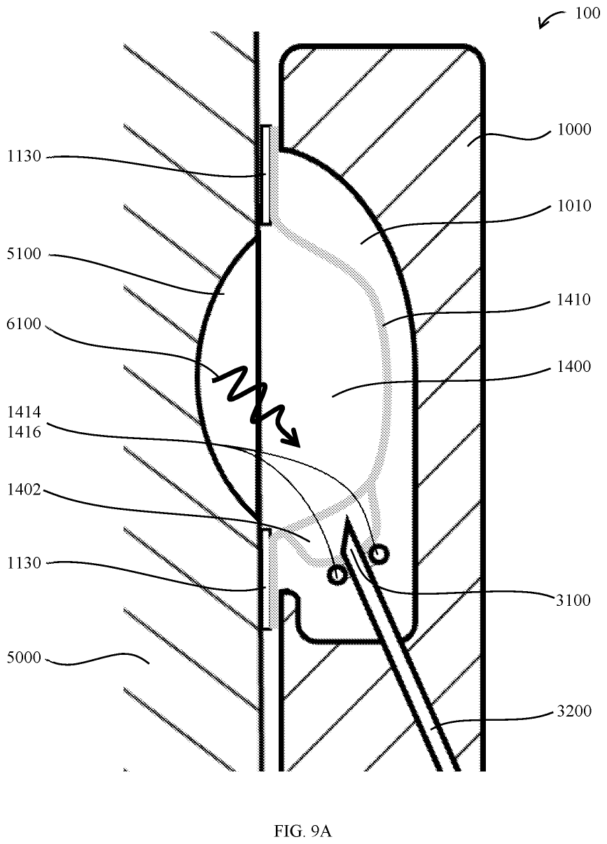

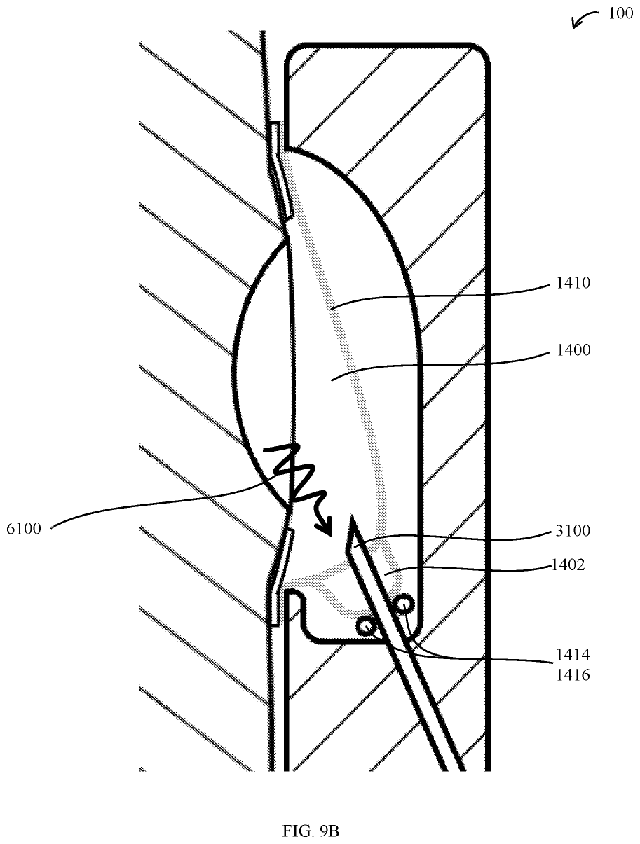

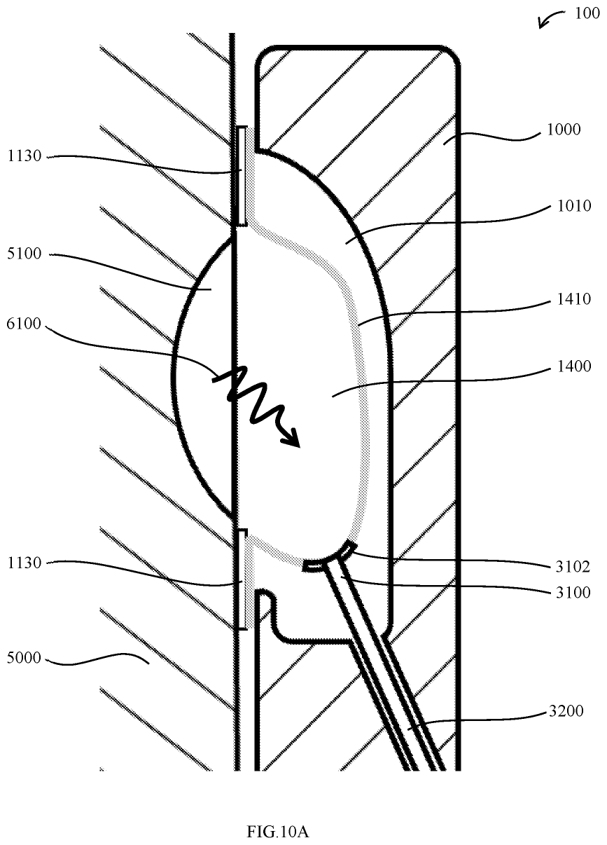

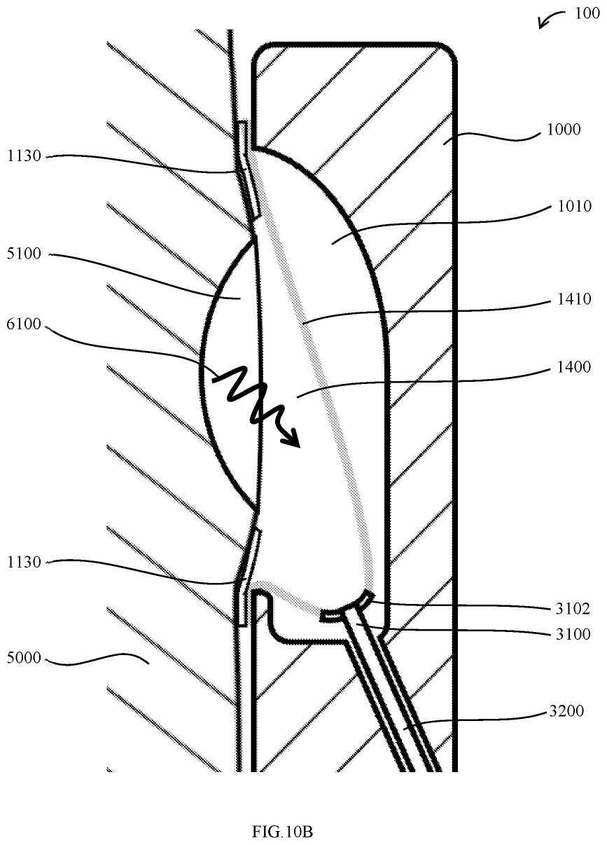

Referring now to FIGS. 9A to 9B and FIGS. 10A to 10B, in an alternative embodiment the blood sampling process includes a waiting step between the laceration of the patient's skin 5000 opening the wound 5100 and the collection of the sampled blood 6100. Such waiting step may have a duration from a few seconds to several minutes, and may be ensured by a timer integrated in the blood sampling device 100, by a timer provided by any electronic device and launched by the user/patient, by a timer included in an app running on the user/patient's smartphone monitoring the blood sampling process, or by the user/patient himself when observing that the wound 5100 is bleeding through a transparent window provided for such purpose within the device structure.

Referring now in particular to FIGS. 9A to 9B, in a first embodiment the suction chamber comprises a first air-tight elastic membrane 1410 attached to the device structure 1000 on its edge, but free to move within a cavity 1010 provided in the structure 1000, and a second air-tight elastic membrane forming a secondary chamber 1402 adjacent to the suction chamber 1400. The secondary chamber 1402 includes an attachment feature 1414 on its external surface. The blood sampling device 100 includes one or more mechanical features 1416 as well-known in micromechanical industry holding the membrane's attachment feature 1414. Before the beginning of the process the suction end 3100 of the needle is protruding in the secondary chamber 1402 and is held air-tight. In this way, the user/patient can connect the vacuum tube 4000 to the needle 3200 before initiating the blood sampling process without risk of losing the vacuum. The suction end of the needle 3200 is sharp enough to be able to pierce the air-tight elastic membrane 1410. At the end of the waiting step of the blood sampling process, the mechanical feature 1416 actuated by the user/patient pulls the membrane 1410 via its attachment feature 1414 and impales it on the suction end 3100 of the needle 3200, opening the path from the patient wound 5100 to the collection tube 4000 via the needle 3200. In a second embodiment, the membrane 1410 may be held in place and the needle 3200 be moved towards the membrane until its suction end 3100 pierces the membrane 1410. As a result, an airtight connection from the vacuum tube 4000 to the suction chamber 1400 via the needle 3200 is established, so the suction chamber 1400 is placed under vacuum, helping to extract the blood from the patient's wound 5100 and allowing the collected blood 6100 to flow into the sampling tube 4000.

Referring now in particular to FIGS. 10A to 10B, in a third embodiment the suction chamber 1400 contains comprises an air-tight elastic membrane 1410 attached to the device structure 1000 on its edge, but free to move within a cavity 1010 provided in the structure 1000. The suction end 3100 of the needle 3200 crosses but remains flush with the internal surface of the elastic membrane 1410 and is attached to the elastic membrane 1410 by means of an elastic, semi-rigid or rigid air-tight fitting 3102. At the end of the waiting step of the blood sampling process, the needle 3200 is actuated by the user/patient towards the sampling tube 4000. As a result, the dispensing end of the needle 3300 pierces the septum 4100 of the vacuum tube 4000 and establishes an airtight connection from the vacuum tube 4000 to the suction chamber 1400 via the needle 3200. Consequently, the suction chamber 1400 is placed under vacuum, helping to extract the blood from the patient's wound 5100 and allowing the collected blood 6100 to flow into the sampling tube 4000. The geometry and the stiffness of the fitting 3102 is selected so as to form a flush funnel with the internal surface of the elastic membrane 1410 when deformed as shown in FIG. 10B, allowing a seamless transition from the suction chamber 1400 to the needle 3000 for the in order to avoid triggering the coagulation of the collected blood 6100 before it reaches the tube 4000.

For all embodiments described in the present application, anticoagulant substance, such as Heparin, on the patient's wound (description FIGS. 7A and 7B) or as a coating within the blood sampling device (description FIG. 8) can be used to slow down the sampled blood coagulation and prevent the clogging of the conduit.

Referring now to FIG. 11, an alternative method to the method 200 using a separate lacerator, for example one that is available on the market, combined with a second or a third embodiment of the blood sampling device is presented:
Step 11a): Optional step, inserting the blood sample vacuum tube in the blood sampling device.
Step 11b): Disinfecting the area of the body where the blood sample will be taken.
Step 11c): Placing the adhesive pad at the localization where the blood sample will be taken.
Step 11d): Placing the lacerator at the localization defined by the adhesive pad and lacerating the skin with the lacerator.
Step 11e): Installing the fluid sampler over the wound, at the localization defined by the adhesive pad. Optionally sticking the fluid sampler on the patient's arm.
Step 11f): Connecting the vacuum tube with the wound: actuating the push-button in case using the second embodiment of the fluid sampling device, or the push button and the lever in case using the third embodiment of the fluid sampling device. In the third embodiment of the fluid sampler, the push button is a safety lock that prevents inadvertent closing of the lever.
Step 11g): Waiting for the vacuum tube to be sufficiently filled. This waiting step may have a duration from a few seconds to several minutes, and may be ensured by a timer integrated in the fluid sampling device, by a timer provided by any electronic device and launched by the user/patient, by a timer included in an app running on the user/patient's smartphone monitoring the fluid sampling process, or by the user/patient himself when observing that the fluid is flowing through a transparent window provided for such purpose within the device structure.
Step 11h): Removing the fluid sampling device from the user/patient.
Step 11i): Folding the dressing part of the adhesive pad over the wound.
Step 11j): Optionally removing the fluid sample tube from the device and closing it.
Step 11k): Sending/bringing the blood sample tube to a lab for analyses.

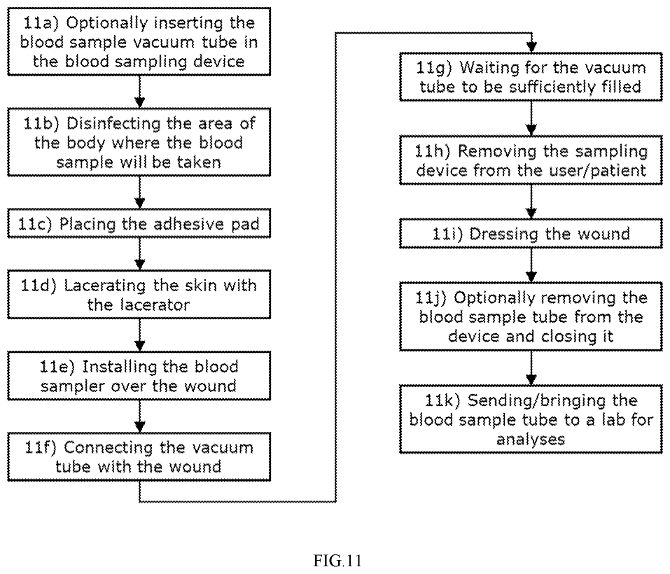

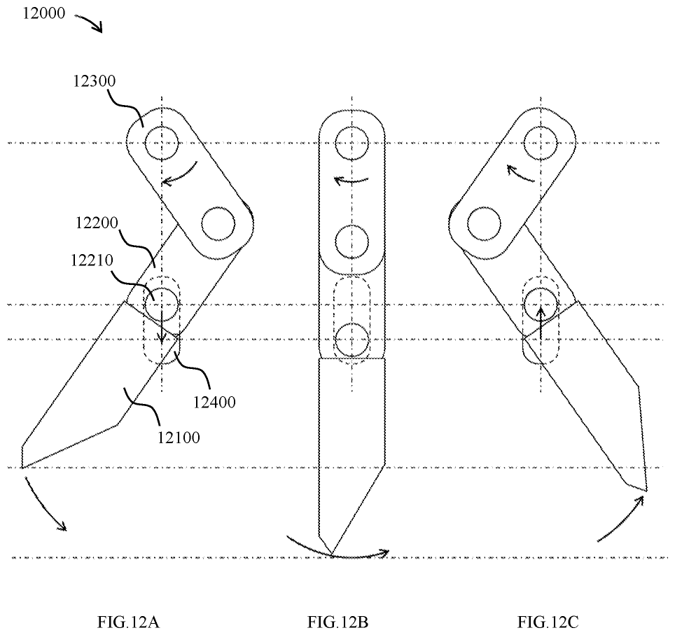

Referring now to FIG. 12A to FIG. 12C, standard lacerators are available on the market. An example of a lacerator that can be used for the process of the present invention is Gentleheel® Heel Incision Device from CardinalHealth, in particular “TGH10X50 Gentleheel®, Toddler, Blue” but other similar incision devices may be used. A lacerator that is appropriate for use in the process of the present invention is able to make a cut in the patient's skin of about 3 mm length for a depth of about 2 mm. Depending on the amount of blood to be collected in a given period of time, the lacerator may have more than one blade to cut more than one wound, making several parallel cuts in one movement. To provide an appropriate wound shape the mechanism 12000 mobilizes the blade 12100 in a combination of a rotation movement and a linear movement. The blade(s) are attached on a lever 12200 that can swivel on a pin 12210, said pin 12210 being free to move within a groove 12400 in the structure of the lacerator. The lever 12200 is actuated via a second lever that is actuated by the user, optionally with a trigger mechanism as well known in the micromechanics industry, optionally with the help of springs. As a result, before the laceration (FIG. 12A), the blade(s) 12100 are kept out of reach of the user/patient, and a safety feature (not shown) prevents inadvertent actuation of the mechanism 12000 by the user/patient. During the laceration (FIG. 12B), the blade(s) 12100 follow the path defined by the mechanism 12000 and open one or more wound(s) in the user/patient's skin. After the laceration (FIG. 12C), the blade(s) are stored away of the reach of the user/patient to avoid inadvertent wounding.

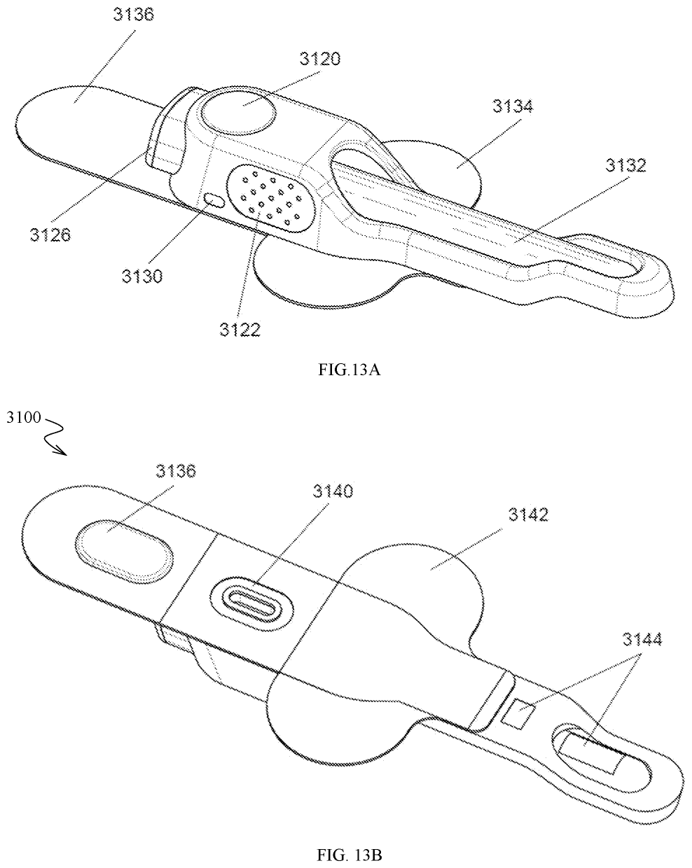

Referring now to FIGS. 13A to 13C The main features of a blood sampling device of the invention are shown. In a first embodiment, the blood sampling device comprises on its top side a grip 3122, a draw blood button 3120, a collect blood button 3126, a flash window 3130 indicating readiness for collecting blood, a blood collection tube 3132, and wing tabs 3134 for device removal as well as dressing 3136 with backer thereon. The blood sampling device 3100 further comprises blood draw reservoir 3140 with wound seal, removal wing tab 3142 with adhesive backing on and machine-readable codes 3144. This first embodiment provides both functionalities of cutting the user/patient's skin and drawing a blood sample. The second embodiment of the device relies on cutting the user/patient's skin with a separate lacerator such as shown in FIG. 12 and the method follows the path described in FIG. 11. Therefore, the second embodiment of the fluid sampling device doesn't have the draw blood button 3120.

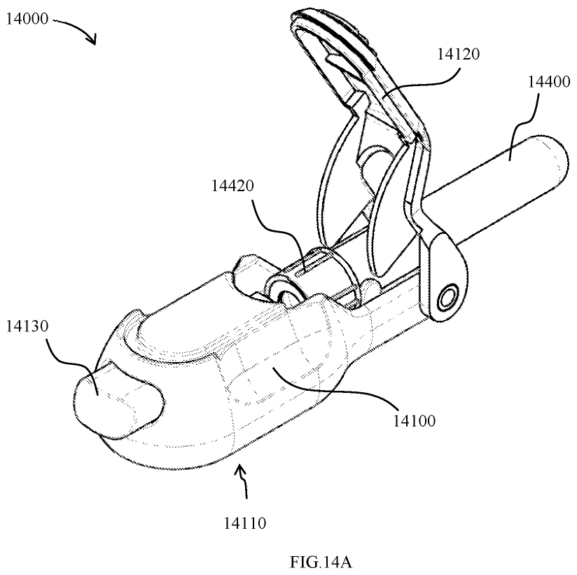

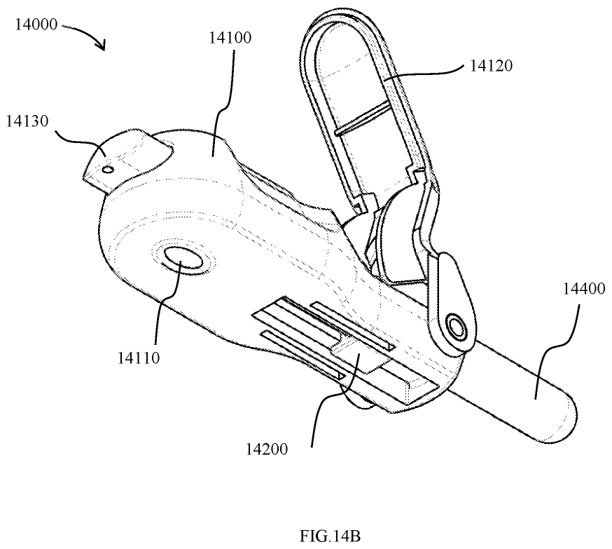

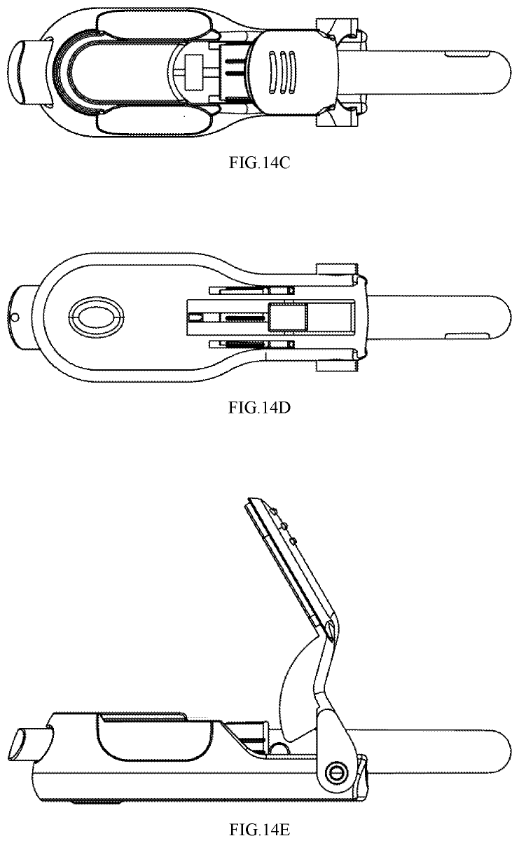

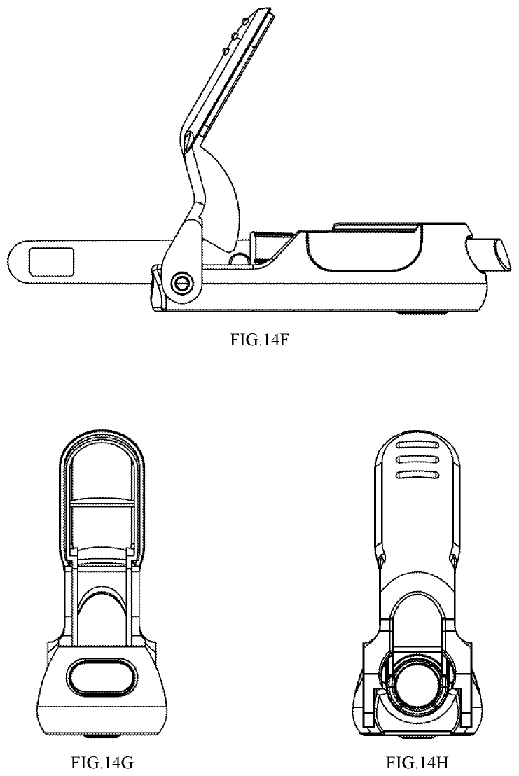

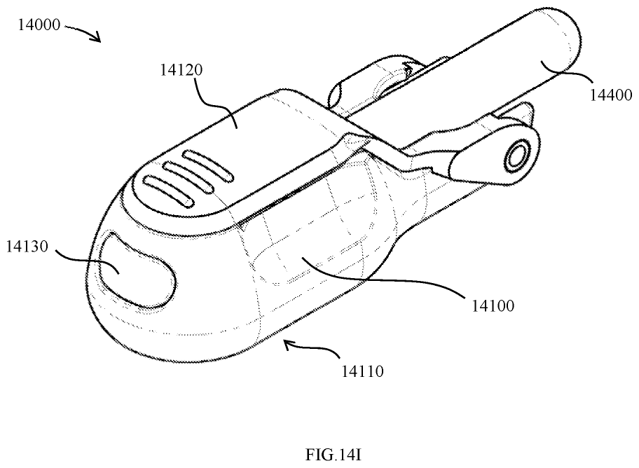

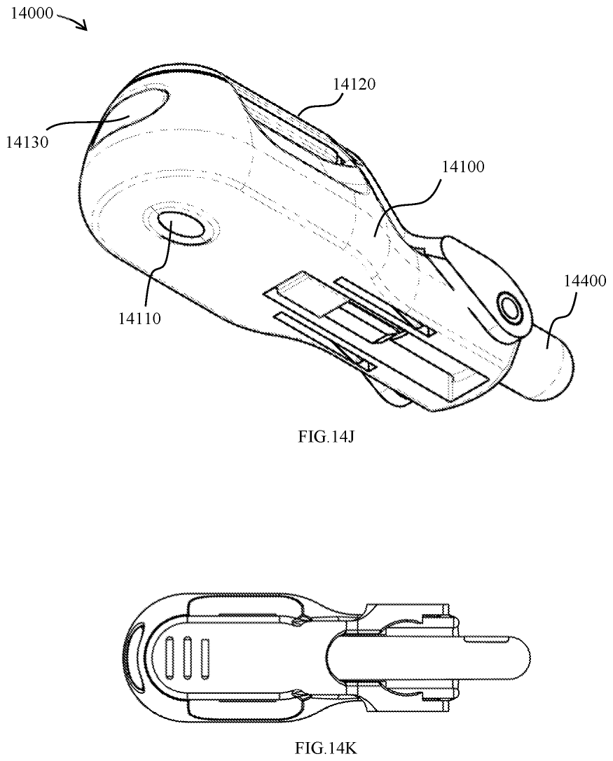

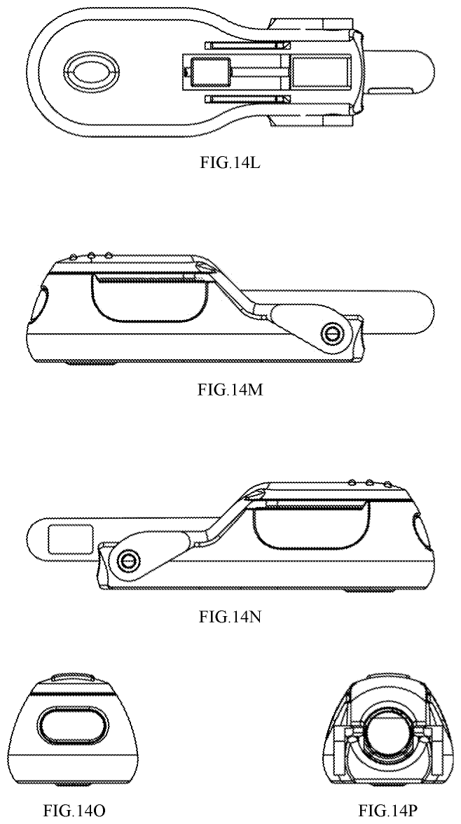

Referring now to FIGS. 14A to 14P, the third embodiment of the device 14000 relies on cutting the user/patient's skin with a separate lacerator and the method follows the schematics of FIG. 11, with a lever 14120 at step 11f. In the third embodiment of the fluid sampling device 14000, the device's body 14100 contains a mechanism 14200 actuated by the user/patient via a lever 14120. The mechanism 14200 holds the vacuum tube 14400 by a mechanical feature 14420, for example the mechanical feature is the lid of the vacuum tube 14400. The vacuum tube 14400 carries a unique identification tag 14430 that can be read by humans and/or by machine means. The lever 14120 is equipped with a safety feature 14130 to avoid accidental activation, so the device 14000 is provided with the vacuum tube 14400 already in place in the device and step 11a) of the method of FIG. 11 is not necessary. FIGS. 14A to 1411 show the fluid sampling device before use, so the lever 14120 is in open position. After the user/patient has followed the steps until having created the wound, the user/patient sticks the fluid sampling device 14000 over the wound so that the suction opening 14110 of the fluid sampling device 14000 is placed over the wound. In the case of blood sampling the user/patient uses the mechanical features provided by the adhesive pad for accurate positioning, but in case of other usages such as for collecting puss or venom the user/patient places the fluid sampling device 14000 visually. The user/patient can see that the suction opening 14110 of the fluid sampling device 14000 is filling with fluid by looking through the fluid sampling device's body 14100 that is made of transparent material, or by looking through a transparent window (not shown) provided in the fluid sampling device's body 14100, or by waiting a predefined duration, this duration may last from a few seconds to several minutes, and may be ensured by a timer integrated in the fluid sampling device 14100, by a timer provided by any electronic device and launched by the user/patient, by a timer included in an app running on the user/patient's smartphone monitoring the fluid sampling process. At this point in time the user/patient releases the safety feature 14130 and actuates the lever 14120, mobilizing the mechanism 14200. The mechanism 14200 moves the vacuum tube 14400 towards the fluid sampling device body 14100, impaling the septum 14110 (not shown) of the vacuum tube 14400 on the conduct, connecting the vacuum tube 14000 to the patient's wound, thereby allowing the collected fluid to flow from the wound to the vacuum tube 14400. Alternatively the mechanism moves the conduct towards the tube 14400 and pierces the septum 14110 (not shown) of the vacuum tube 14400, connecting the vacuum tube 14000 to the patient's wound, thereby allowing the collected fluid to flow from the wound to the vacuum tube 14400. The configuration of the fluid sampling device 14000 is made so that the user/patient pushes on the lever 14120 over the localization of the wound so as to not risk unsticking the fluid sampling device 14000 from the patient's skin. FIGS. 14I to 14P show the fluid sampling device 14000 after the lever 14120 has been pushed.

Referring now to FIGS. 15A to 15B, an adhesive pad 15000 is provided with at least one mechanical feature that allows for the precise placement of the lacerator and of the fluid sampling device. The adhesive pad 15000 includes an adhesive zone 15100 for its attachment to the patient's skin, one or more visual target and mechanical features 15200 allowing the precise positioning of the lacerator and of the fluid sampling device, and a dressing part 15300 for covering the wound after the fluid sampling process. The target and mechanical feature(s) 15200 may have the shape of a ridge against which the user/patient can push the lacerator and the fluid sampling device. Before the fluid sampling process starts, the adhesive zone 15100 is covered with backers 15110, the target and mechanical positioning feature(s) 15200 is covered with backers 15210 and the dressing part 15300 with backer 15310. The user/patient removes the respective backers as appropriate in order to follow the fluid sampling process.

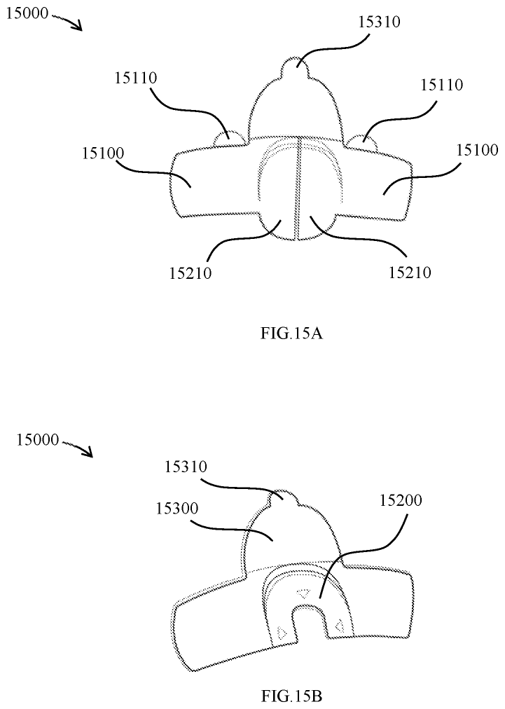

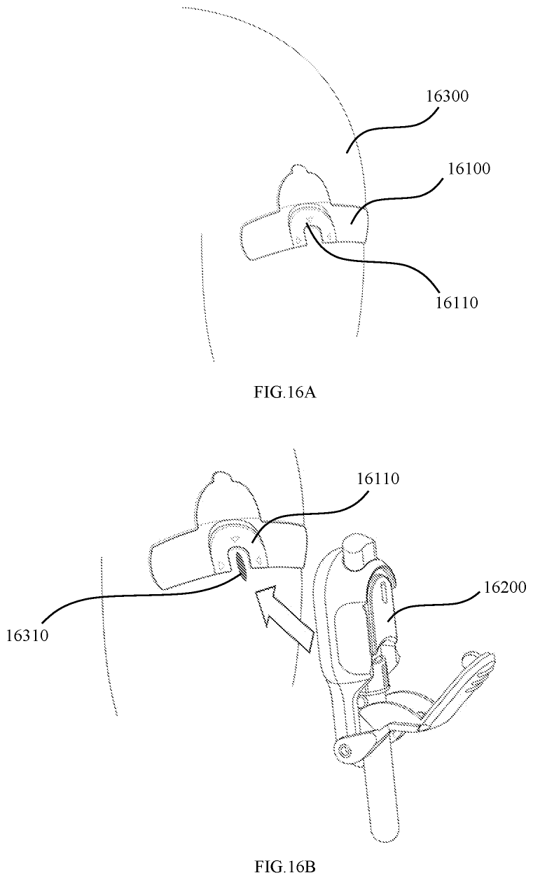

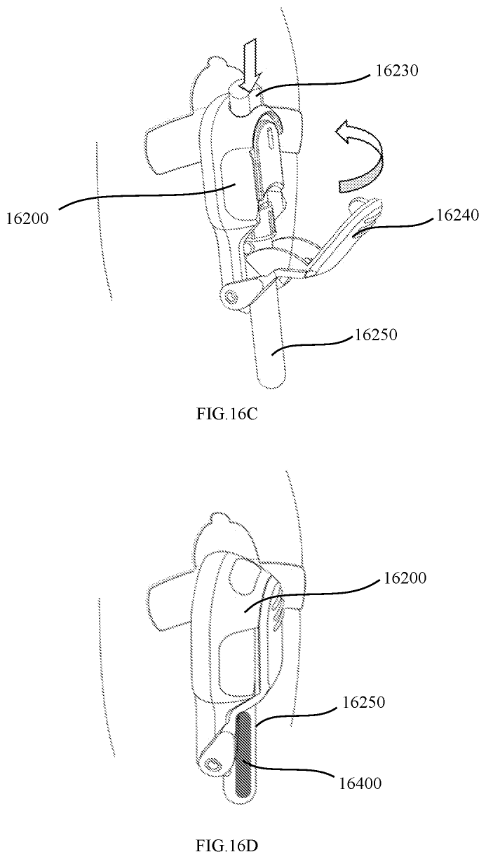

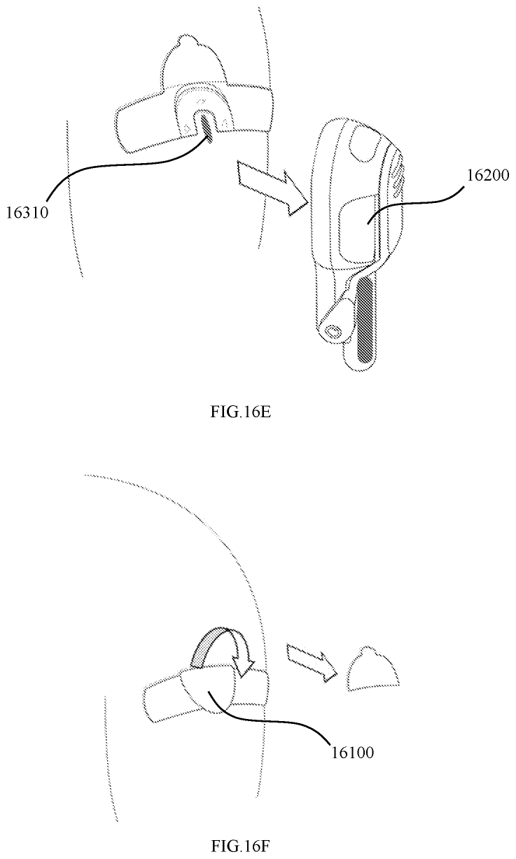

Referring now to FIGS. 16A to 16F, some key steps of the method using the adhesive pad 16100, a lacerator and the fluid sampling device 16200 of the invention to sample blood on the arm 16300 of a patient are shown.

FIG. 16A illustrates the step of installing the adhesive pad 16100 on the arm 16300 of the patient and exposing the target and mechanical feature 16110.

FIG. 16B illustrates the step of installing the fluid sampling device 16200 using the target and mechanical feature 16110 over the wound 16310 after it has been cut open with the lacerator.

FIG. 16C illustrates the step of activating the sampling with the fluid sampling device by pushing the safety button 16230 and then closing the lever 16240.

FIG. 16D illustrates the step of waiting and monitoring the fluid 16400 flowing into the vacuum tube 16250 of the fluid sampling device.

FIG. 16E illustrates the step of removing the fluid sampling device 16200 after sampling.

FIG. 16F illustrates the step of dressing the wound 16310 with the adhesive pad 16100.

Referring now to FIG. 17, the geometry of the suction chamber 17100 of the fluid sampling device 17000 of the invention is adapted to provide an airtight interface at the suction opening to the patient's skin and to allow the collecting and free flowing of the sampled fluid through the conduct 17300 into the vacuum tube. The internal volume of the suction chamber 17100 is as small as possible to avoid unnecessary losses of vacuum and stagnation of the sampled fluid. The internal surface 17110 of the suction chamber 17100 and the internal surface 17310 of the conduct are made as slick and flush as possible, optionally coated with an antiadhesive coating, optionally coated with an anticoagulant coating, to minimize the risk of coagulation of the sampled fluid and to promote an as free as possible flow of the sampled fluid through the suction chamber 17100, through the conduct 17300 and into the vacuum tube. The edge 17210 of the suction opening 17200 is made with a concave shape, so as to avoid any of the sampled fluid to stagnate at the suction opening 17200 and/or to slip below the sampling device 17000. The top side 17120 and the bottom side 17130 of the suction chamber 17100 form together a funnel leading from the suction opening 17200 to the conduct 17300. The bottom side 17130 of the suction chamber 17100 is made so as to present a downwards slope relative to gravity when the device 17000 is placed on the arm of the patient.

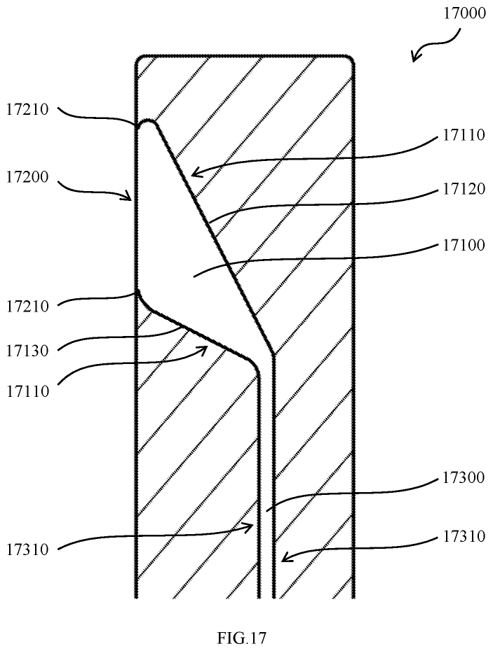

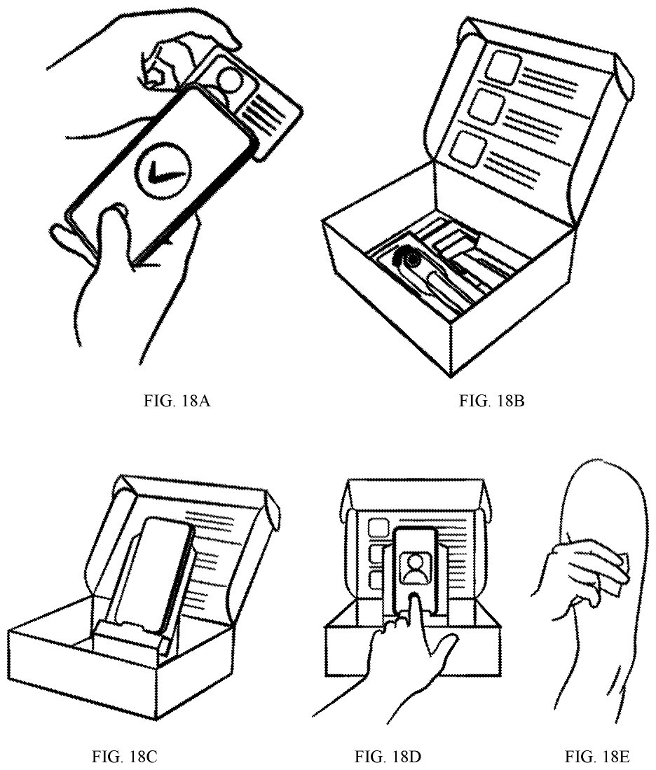

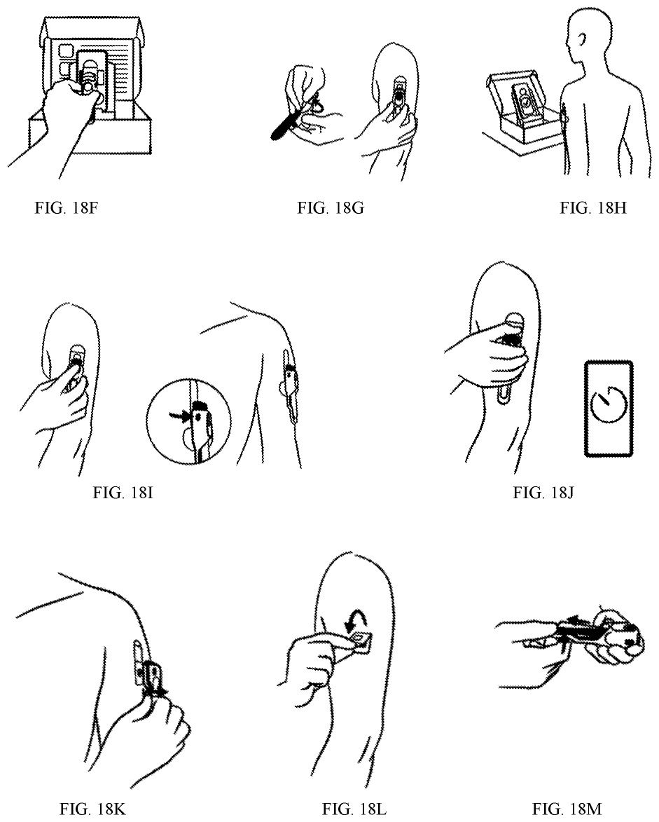

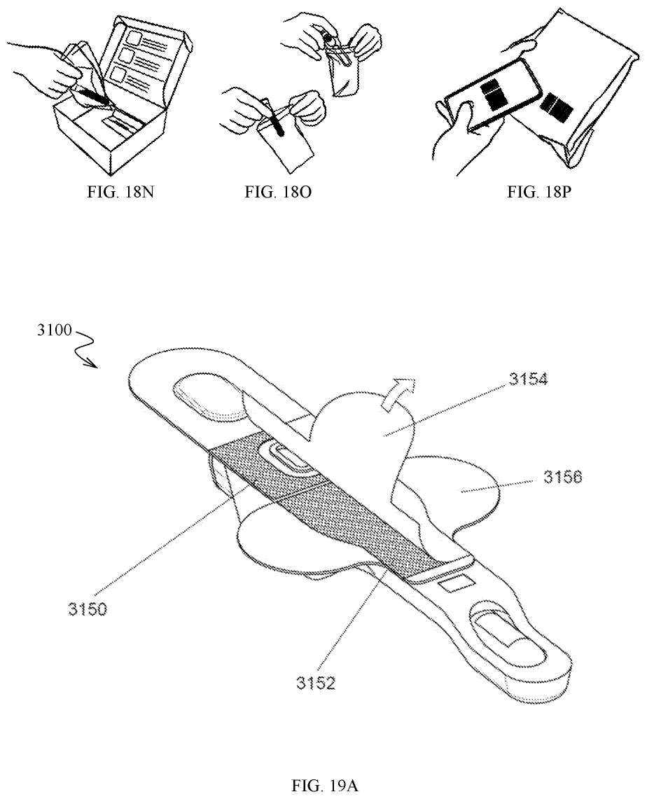

Referring now to FIGS. 18A to 18P, a fourth embodiment of the method of using the system of the invention is shown, and the specific features of the user authentication system 1900, of the device authentication system 1902, of the capillary blood sampling device 3100, 4100 and of the adhesive integrated dressing 3174 included in the capillary blood sampling device 3100, can be deducted.

FIG. 18A shows step 1 of the fourth embodiment of the method of using the system of the invention. Step 1 comprises: a) loading a dedicated App for use with the invention via scan web site or prescription; b) executing a consent form; c) entering demographics information; d) photographing government issued ID or another identification means; and e) taking a selfie for association with the patient and the device as well as the sample taken.

FIG. 18B shows step 2 of the fourth embodiment of the method of using the system of the invention. Step 2 comprises: a) opening package; b) reading instructions; c) removing contents from package; d) scanning QR code on kit box to launch App and guide; e) checking contents; and f) cleaning phone with sanitary wipe included in kit.

FIG. 18C shows step 3 of the fourth embodiment of the method of using the system of the invention. The step 3 comprises: a) deploying integrated phone stand in box; b) wiping down phone with included sanitary towelette; and c) washing hands.

FIG. 18D shows the step 4 of the fourth embodiment of the method of using the system of the invention. Step 4 comprises: a) taking a selfie with phone on stand; b) starting video of self with face and arm in the frame.

FIG. 18E shows the step 5 of the fourth embodiment of the method of using the system of the invention. Step 5 comprises: a) following App guide (reading text/reviewing figures); b) preparing arm with a warm towel; c) opening alcohol towelette from kit; and d) wiping site on upper arm with alcohol, then letting dry.

FIG. 18F shows step 6 of the fourth embodiment of the method of using the system of the invention. step 6 comprises: a) removing device from kit packaging; and b) presenting QR code on device and tube to camera on stand.

FIG. 18G shows step 7 of the fourth embodiment of the method of using the system of the invention. Step 7 comprises: a) removing adhesive backing; and b) adhering device to upper arm.

FIG. 18H shows step 8 of the fourth embodiment of the method of using the system of the invention. Step 8 comprises: confirming video of self with face device are in the frame.

FIG. 18I shows the step 9 of the fourth embodiment of the method of using the system of the invention. Step 9 comprises: a) pushing a first button; b) waiting until in flash window an indication is indicated; c) confirm the indication; and d) if no indication is indicated in prescribed time, follow guide to return kit.

FIG. 18J shows the step 10 of the fourth embodiment of the method of using the system of the invention. Step 10 comprises: a) pushing a second button; and b) waiting for timer on App to indicate tube is full with a means of communication progression.

FIG. 18K shows the step 11 of the fourth embodiment of the method of using the system of the invention. Step 11 comprises: a) pulling on wing tabs to peel the device off the arm; b) leaving dressing on arm; and c) returning device to kit box.

FIG. 18L shows the step 12 of the fourth embodiment of the method of using the system of the invention. Step 12 comprises: a) removing dressing backer revealing gauze pad and adhesive; and b) folding gauze dressing down over wound.

FIG. 18M shows the step 13 of the fourth embodiment of the method of using the system of the invention. Step 13 comprises: a) popping out end of vial from device; and b) pulling vial out of device.

FIG. 18N shows the step 14 of the fourth embodiment of the method of using the system of the invention. Step 14 comprises: a) securing sample vial in biohazard pouch from kit; and b) securing device in separate biohazard pouch from kit.

FIG. 18O shows the step 15 of the fourth embodiment of the method of using the system of the invention. Step 15 comprises: a) stopping video; b) removing phone from kit stand; c) collapsing phone stand; and d) securing both biohazard pouches in kit box.

FIG. 18P shows the step 16 of the fourth embodiment of the method of using the system of the invention. Step 16 comprises: a) sealing kit box in return shipping pouch; b) scanning QR code on shipment package; c) mailing package; d) confirming shipment; e) App notifies patient with test results.

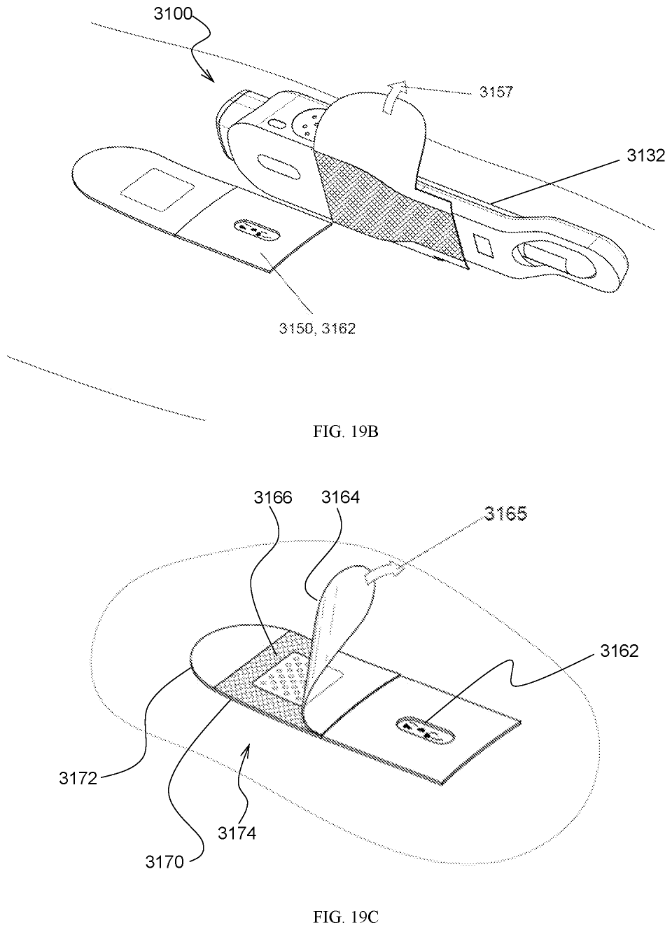

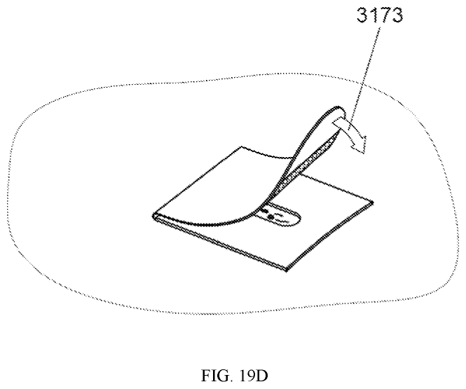

Referring now to FIGS. 19A to 19D, more details about the specific features of the adhesive integrated dressing 3174 included in the capillary blood sampling device 3100 of the invention are shown. The blood sampling device 3100 comprises an adhesive 3150 around wound site separate from the adhesive 3152 on the bottom of the device. The blood sampling device further comprises an adhesive backing 3154 which is removably located on the adhesive 3150, 3152, the adhesive backing 3154 is removed prior to the device being applied to a patient's body. Once the device is placed on a patient's arm, the device may be pulled up 3157 on a wing tab 3156 to peel the device off of the patient's skin after tube 3132 is filled. The adhesive 3150 surrounding the wound site remains behind on skin with an encompassing features to restrain the blood from flowing out of the exposed site area temporarily. Then, a dressing backer 3164 is peeled off 3165 so as to reveal gauze pad 3166 surrounded by adhesive 3170. The dressing 3174 comprise a dressing tab 3172 without adhesive. The dressing tab 3172 is pullable 3173 to fold the gauze pad onto the wound site and may be affixed to cover the wound.

As a result, the adhesive integrated dressing 3174 is adapted to ensure the attachment of the blood sampling device 3100 to the patient's skin, the air-tightness between the patient's skin and the blood sampling device 3100 during the sampling process, and the dressing of the wound after the sampling process.

Capillary blood sampling devices for non-medically trained users of the current art usually create the wound in the patient's skin by perforation with one or more needles, or even without perforation, which only allows for the collection of relatively small volumes of fluid, typically less than 150 μl in 5-10 minutes.

In another embodiment, the present invention provides means for the capillary blood sampling device 10, 1100, 2010, 2210, 3100, 4100 to make a significantly larger cut than what is usually known in existing capillary blood sampling devices, so that a significantly larger amount of blood, typically more than 500 μl, preferably 1 ml can be collected over a reasonable period of time, typically less than 15 minutes, preferably less than 10 minutes. The capillary blood sampling device of the invention uses one or more cutting blade(s) 1004 (e.g., but not limited to cutting blades 302, 5450, 5456, 3260, 3360, 3460), instead of one or more needle(s), so that the user/patient's skin is lacerated instead of punctured. For the purpose of the present disclosure, the functioning of the sampling device of the invention is described assuming it contains one cutting blade, but it must be understood that the sampling device of the invention may contain more than one cutting blade in order to increase the amount of blood collected and/or to decrease the blood collection time. Moreover, the current invention provides cutting solutions that favor a quick healing of the wound after the blood sampling is complete. The purpose of the invention is therefore to create a wound in the user/patient's skin that has an optimal depth for cutting as many capillaries as possible, while avoiding unnecessary wound width and length so that the natural healing of the wound can happen as fast as possible after the blood collection. The ideal cutting depth may vary as function of the patient's age, gender, ethnical group and/or health condition, as a result several adapted versions of sampling devices may be provided. Typically ideal cutting depth is between 1 mm and 2 mm.

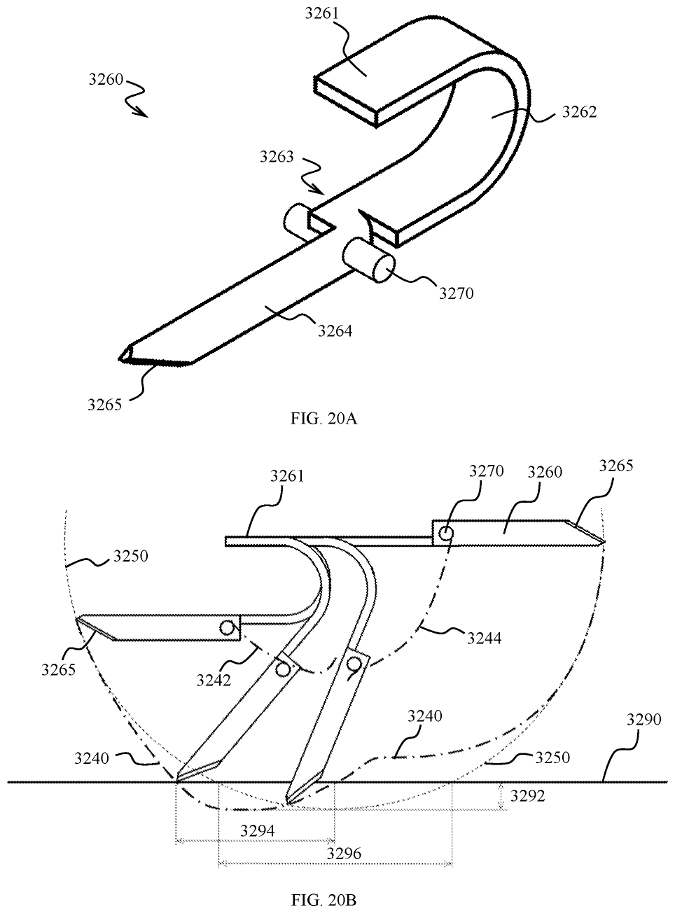

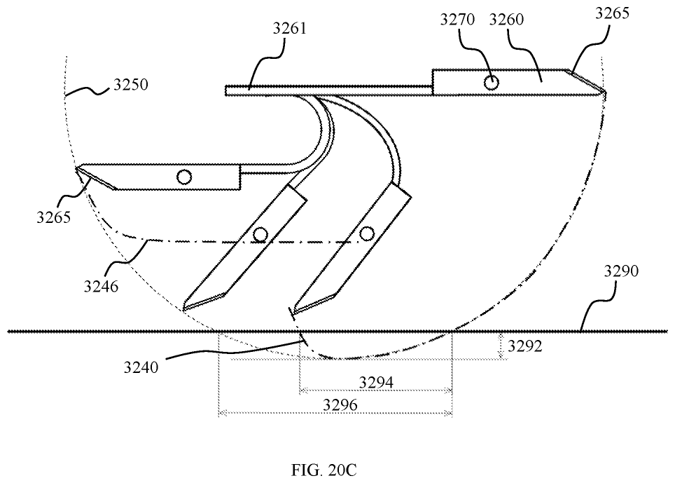

Referring now to FIGS. 20A to 20C, in a fourth embodiment the cutting blade 3260 of a device of the invention is made of a single part, typically out of sheet metal or spring steel, but may be also made of other materials, including composite materials presenting the appropriate mechanical properties. The cutting blade 3260 has an end 3261 facing the sampling device structure so that it can be attached to it, followed by an elastic zone 3262 which is bent elastically when the cutting blade is ready to be used. In this way, the cutting blade 3260 contains all the energy necessary for its movement, and the user/patient only needs to release it by depressing the draw blood button 3120. The elastic zone 3262 has an appropriate, preferably flat cross-section so as to provide a preferred release trajectory perpendicular to its attachment end 3261. In one embodiment, after the elastic zone 3262, the cutting blade is twisted by 90° in the area 3263 so as to provide a blade section 3264 which is in the same plane as the release trajectory. The blade section 3264 has a high rigidity in the direction of the release trajectory.

The elastic zone may take the form of a multiturn torsion spring, such as can be found in clothes pins, in such case the cross-section of the blade is round and the varying stiffnesses necessary for the function are obtained by variation of the thermal treatments of the different zones of the blade. Towards the end of the blade section 3264, a cutting edge 3265 is provided so as the lacerate the user/patient's skin 3290. After completion of the movement of the cutting, edge 3265 faces away from the user/patient's reach. Optionally the cutting blade 3260 includes a finger 3270 that interacts with the device's structure elements 3242, 3244, 3246 in order to bias the natural release trajectory 3250 of the cutting blade 3260 when released to obtain a modified trajectory 3240.

Typically, the natural release trajectory 3250 of the cutting blade 3260 is substantially circular, elliptical or spiral. As a result, the laceration in the patient's skin 3290 is substantially circular and with a relatively large radius, and the wound length 3296 is relatively long for a small portion at the desired depth 3292. When elements of the device's structure 3242, 3244, 3246 interact with the finger 3270 of the cutting blade 3260, the resulting trajectory 3240 of the cutting blade 3260 is modified so that the resulting laceration of the patient's skin 3290 has a steeper dive and retraction path, resulting in a shorter wound length 3294 for a longer proportion of the wound at the desired depth 3292. The modified trajectory 3240 allows for a larger volume of capillary blood to be collected, for a globally smaller wound, favoring a quicker healing of the wound after the blood collection.

Referring now to FIG. 20B, as an example, the element of the device's structure 3242 is made to locally extend the radius of natural release trajectory 3250, and the element of the device's structure 3244 is made to locally shrink the radius of the natural release trajectory 3250.

Referring now to FIG. 20C, a further example the element of the device's structure 3246 is made to locally shrink the radius of natural release trajectory 3250.

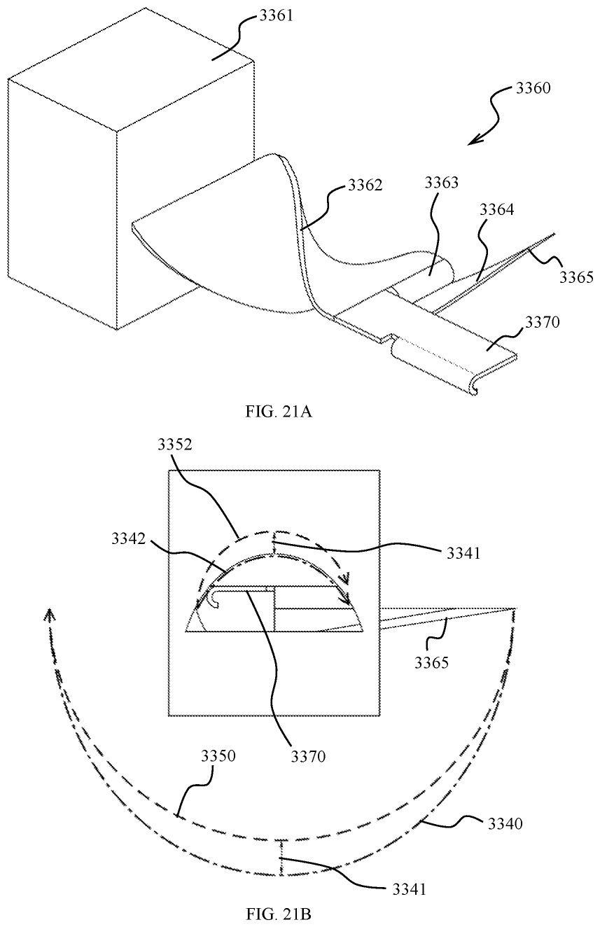

Referring now to FIGS. 21A to 21B, in a fifth embodiment the cutting blade 3360 of a device of the invention is made of a single part, typically out of sheet metal, but may be also made of other materials, including composite materials presenting the appropriate mechanical properties. The cutting blade 3360 has an end 3361 facing the sampling device structure so that it can be attached to it, followed by an elastic zone 3362 which is twisted elastically when the cutting blade is ready to be used. In this way, the cutting blade 3360 contains all the energy necessary for its movement, and the user/patient only needs to release it by depressing the draw blood button 3120. The elastic zone 3362 has an appropriate, preferably flat cross-section so as to provide a preferred, planar natural release trajectory 3350. In one embodiment, after the elastic zone 3362, the cutting blade is twisted by 90° in the area 3363 so as to provide a blade section 3364 which is in the same plane as the release trajectory. The blade section 3364 has a high rigidity in the direction of the release trajectory. Towards the end of the blade section 3364, a cutting edge 3365 is provided so as to lacerate the user/patient's skin (not represented). After completion of the movement, the cutting edge 3365 faces away from the user/patient's reach. Optionally, the cutting blade 3360 includes a finger 3370 that interacts with the device's structure element 3342, in order to bias the natural release trajectory 3350 of the cutting blade 3360 when released, generating a modified release trajectory 3340.

Typically the natural release trajectory 3350 of the cutting blade 3360 is substantially circular or spiral. As a result, the laceration in the patient's skin is substantially circular and with a relatively large radius, and the wound length is relatively long for a relatively small portion at the desired depth. When an element of the device's structure 3342 interacts with the finger 3370, the resulting trajectory 3340 of the cutting blade 3360 can be modified so that the resulting laceration of the patient's skin has a steeper dive and retraction path, resulting in a shorter wound length, and a longer proportion of the wound at the desired depth. The modified trajectory 3340 allows for a larger amount of blood volume to be collected, for a generally smaller wound, favoring a quicker healing of the wound after the blood collection. As an example, the element of the device's structure 3342 is made to shrink locally the radius 3352 of the natural release trajectory by an offset 3341 on a part of the release trajectory 3340. In addition, the element of the device's structure 3342 can be made with a more complex shape so as to provide a more elaborated modified trajectory 3340.

Referring now to FIGS. 22A to 22C, in a fourth embodiment the cutting blade 3460 of a device of the invention is made of a single part, typically out of sheet metal, but may be also made of other materials, including composite materials presenting the appropriate mechanical properties. Referring now to FIG. 22A, the cutting blade 3460 has a rotative attachment 3466 to the device's structure and a cutting edge 3465, which is kept in retracted position prior to the cutting process.

Referring now to FIG. 22B, when actuated the cutting blade 3460 engages into the patient's skin 3490 in a rotative movement, but the rotative movement is limited by the structure so that the patient's skin is not completely lacerated.

Referring now to FIG. 22C, after the laceration the cutting blade 3460 is retracted in reverse rotative movement, leaving a wound under the patient's skin that is much smaller than if the laceration had been made completely. As a result, a larger amount of blood volume can be collected thanks to the deep laceration, but at the surface of the patient's skin 490, the opening is smaller, favoring a quicker healing of the wound after the blood collection.

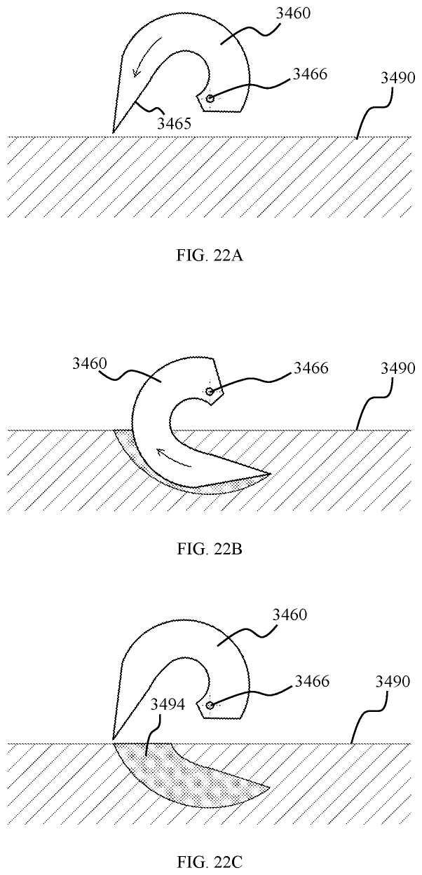

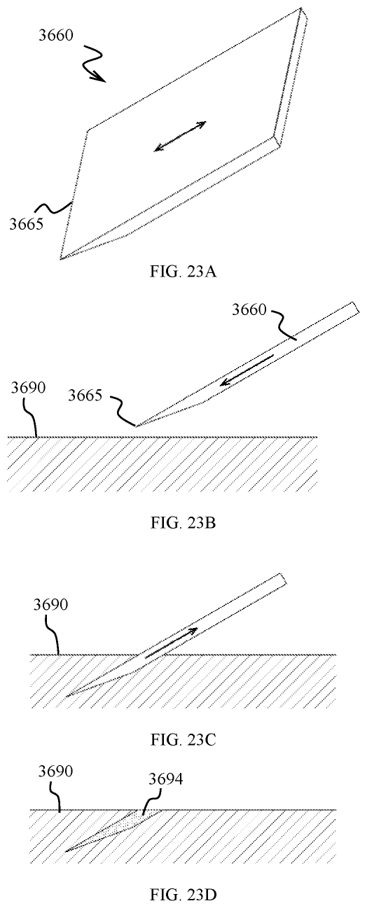

Referring now to FIGS. 23A to 23D, in a fifth embodiment the cutting blade 3660 of a device of the invention may be a standard scalpel blade or any other rigid blade, typically out of sheet metal, but may be also made of other materials, including composite materials presenting the appropriate mechanical properties. The cutting blade 3660 has a cutting edge 3665, positioned substantially parallel to the skin of the user/patient 3690. The cutting blade is guided in a linear movement by the device's mechanism (not shown) in order to penetrate the user/patient's skin at a substantially non-orthogonal angle (FIG. 23B). In this way, the cutting edge 3665 penetrates completely in the skin of the user/patient, reaching a substantially uniform depth along all its length, creating a substantially rectangular wound, oriented non-orthogonally in the user/patient's skin (FIG. 23C). When the cutting blade is retracted (FIG. 23D), the flap generated by the non-orthogonal wound closes naturally the wound's entrance, favoring a quicker healing of the wound after the blood collection.

In another embodiment, the invention provides for verification of self-administered medical processes. An objective of this embodiment is to make sure the user/patient is identified and to make sure the blood in the sampling tube(s) is the blood of this patient

What may be at stake here is:
Reliable blood sampling process;
Authorization to work, to travel, to be in contact with family in times of epidemics;
Authorization to use a certain treatment;
Automatized detection of epidemics by large-scale sampling programs;
Verification of the efficiency of a given treatment (payment subject to treatment success);

Still further, payment of a treatment may be at stake, contingent on vaccination and only if fully administered. Therefore, making a secure patient & process verification is crucial.

In another aspect, verification of self-administered medical processes is important. For example, use of a smartphone application may be made in order to make a video (or a time-lapse) where the patient's face as well as the process itself are visible in the video all along the process duration. A comparison of the patient's ID with the patient's face may be made. The patient's face recognition and whole process may be observed. The system could be configured to launch the process only when all ID's are confirmed. Automatized handling of the logistics (sampling tube collection & transport, treatment re-supply, etc.) may be implemented. In the case that the smartphone app is configured to analyze the video in real-time, step-by-step instructions may be provided to the patient in real-time, while the process is being executed. Other features could be included such as automated monitoring of process performance. Despite the potential, there will always be challenges to deal with, particularly in degraded conditions, when dealing with insufficient network coverage at the moment of running the process. Optionally, temporary network coverage may be provided via drones/balloons for the duration of the treatment campaign. It would be best however, if the App is able to run independently of network coverage. Saving all data on the smartphone and making verifications at a later stage may also be desirable. Respecting private data regulations is an issue of course. The App may be configured to run a first analysis and then an encryption routine to make private data unreadable, with full video optionally saved for later use in case of need (e.g. formal proof in a court).

Main components of the system include a smartphone or laptop/computer or similar and camera (may be included in the smartphone/laptop/computer), the application to be run by the smartphone or laptop/computer; the device for running the injection/sampling process, which may be re-usable. In addition, the treatment to be injected/one or more empty tubes/vials to contain the collected blood sample(s) (may be under vacuum).

As for some key features of the system of the invention, the app must be able to read treatment container/sampling tube's unique ID using for example a standard bar-code/QR-code. The App must be able to identify key process steps. The device may need to include automatized wireless signal emission (information: ID/process started/process ongoing/process finished/error). Such signal may be visible (e.g. blinking/colored LEDs) for easy interpretation in the video. The device of the invention may optionally include visible features/landmarks for easier orientation verification in the video. The App should be able to launch the treatment process. The device is optionally equipped with remote triggering feature and advantageously includes a unique ID.

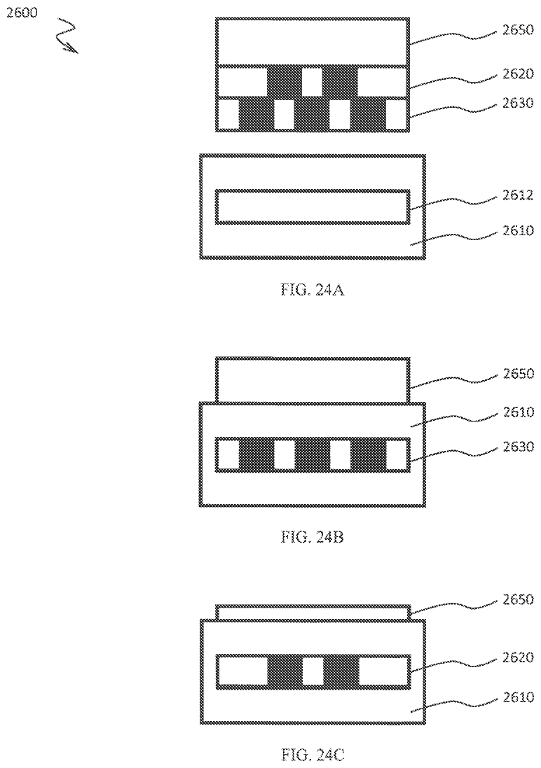

Referring now to FIGS. 24A to 24D, visible features 2600 integrated in the device 2610 of the invention that can be recognized easily may optionally take the form of the combination of different high-contrast patterns 2620, 2630 applied on a mobile part (in this example a button 2650) and visible through a window 2612 only one at a time. As a result, predefined positions of the mobile part (button 2650 in high position in FIG. 24B, button 2650 in low position in FIG. 24D) can be easily recognized by the human observer, or easily identified by an image analysis software. Colored parts and/or features may also protrude and/or disappear from/in the device at specific steps of the process so as to materialize the completion of the step and be easily recognized by the human observer, or easily identified by an image analysis software.

For the purpose of the present disclosure the words blade, cutting blade or lancet are used indifferently and must be understood as equivalents.

The invention may have other uses. For example, it may be applied to another medical treatment other than blood sampling (the device may be an injection device, a pills distributor). It may be applied to filling a voting form at home, signing documents, proving one's ID during a teleconference, or taking a remote exam.

Note that in this application, where ever blood sampling is mentioned, it should be understood that puss or venom can be substituted for blood.

The invention can be summarized as including the following feature sets:
1. A disposable fluid sampling device including:
(a) body fluid sampling means, optionally body fluid sampling means for auto-sampling;
(b) optionally, analysis means configured to use one or more droplet(s) of the sampled fluid to analyze the fluid; and
(c) an interface for a medical analysis tube meeting size and interface standards, the tube adapted to be filled with a sample of fluid for analysis in a point of care or medical lab, wherein the device includes an interface for and a vacuum tube, the vacuum tube adapted to provide suction necessary to fill the vacuum tube with the fluid.
2. A method of using the fluid sampling device of feature set 1, wherein, in a first step, a test subject is tested for a pathogen, and, if tested positive, in a second step, a treatment and/or quarantine protocol is initiated to ensure that a test subject having a positive test is handled in a manner which helps minimize the spread of the pathogen.
3. A lancet adapted for making a laceration in the skin of a user/patient for a disposable fluid sampling device of feature set 1 used for collecting a body fluid, wherein the lancet construction is made in one piece of material and is disposed in a holder so as to enable its providing of the energy for movement and the guiding of its movement.
4. The sampling device of feature set 1, wherein a fluid extraction passage or conduit from the patient's wound to the vacuum tube is formed in a needle.
5. The sampling device of feature set 1, wherein the user is a non-medically trained user.
6. The sampling device of feature set 1, wherein the fluid reservoir is the vacuum reservoir.
7. The sampling device of feature set 1, wherein the fluid is, at least in part, blood.
8. The sampling device of feature set 1, wherein the fluid is, at least in part, puss.
9. The sampling device of feature set 1, wherein the fluid is, at least in part, venom.
10. The sampling device of feature set 1, wherein, optionally, the device is adapted to use one or more droplet(s) of the sampled body fluid, to analyze the body fluid.
11. The sampling device of feature set 1, wherein the device includes a vacuum tube and an interface therefor, the vacuum tube providing suction necessary to fill the vacuum tube with the body fluid.
12. The sampling device of one of feature sets 1, 4 to 11, wherein the medical analysis tube is a standard medical analysis tube.
13. A method of capillary blood sampling, optionally self sampling, the method includes at least the steps of:
   a) placing an adhesive pad at the intended location for the capillary blood sampling, the adhesive pad including at least one mechanical localization feature and optionally visual indications;
   b) lacerating the user/patient's skin with a lacerator, thereby opening at least one wound in the user/patient's skin, the lacerator being positioned using the at least one mechanical localization feature of the adhesive pad;
   c) collecting the patient's capillary blood with a fluid sampling device, the device being positioned on the at least one wound using the at least one mechanical localization feature of the adhesive pad and the device using vacuum to collect blood from the at least one wound and to fill a sample analysis tube; and
   d) dressing the at least one wound using a foldable part of the adhesive pad.
14. The device of feature set 1, wherein the device further includes an adhesive pad, the adhesive pad adapted for application at the intended location for the capillary blood sampling, the adhesive pad including at least one mechanical localization feature and optionally visual indications, the adhesive pad further containing at least one mechanical feature that allows for the precise placement of the lacerator and of the fluid sampling device and a foldable portion adapted for covering the at least one wound after the blood collection.
15. The device of feature set 1 or 14, wherein the device further includes a lacerator including a lancet adapted for making a one or more lacerations in the skin of a user/patient, wherein the lancet construction is made in one piece of material and provides the energy and the guiding for its movement.
16. The device of feature set 1, 14 or 15, wherein the device further includes a lacerator including a lancet adapted for making a one or more lacerations in the skin of a user/patient, wherein the lacerator contains one blade for each of the one or more lacerations and makes the one or more lacerations simultaneously.
17. The lacerator of feature set 16, wherein when lacerating the patient's skin the trajectory of the at least one blades follows a trajectory that combines at least one rotary and at least one linear component of movement.

Further, the invention should be considered as comprising all possible combinations of every feature described in the instant specification, appended claims, and/or drawing figures which may be considered new, inventive and industrially applicable.

It should be appreciated that the particular implementations shown and herein described are representative of the invention and its best mode and are not intended to limit the scope of the present invention in any way.

As will be appreciated by skilled artisans, the present invention may be embodied as a system, a device, or a method.

Moreover, the system contemplates the use, sale and/or distribution of any goods, services or information having similar functionality described herein.

The specification and figures should be considered in an illustrative manner, rather than a restrictive one and all modifications described herein are intended to be included within the scope of the invention claimed. Accordingly, the scope of the invention should be determined by the appended claims (as they currently exist or as later amended or added, and their legal equivalents) rather than by merely the examples described above. Steps recited in any method or process claims, unless otherwise expressly stated, may be executed in any order and are not limited to the specific order presented in any claim. Further, the elements and/or components recited in apparatus claims may be assembled or otherwise functionally configured in a variety of permutations to produce substantially the same result as the present invention. Consequently, the invention should not be interpreted as being limited to the specific configuration recited in the claims.

Benefits, other advantages and solutions mentioned herein are not to be construed as critical, required or essential features or components of any or all the claims.

As used herein, the terms “comprises”, “comprising”, or variations thereof, are intended to refer to a non-exclusive listing of elements, such that any apparatus, process, method, article, or composition of the invention that comprises a list of elements, that does not include only those elements recited, but may also include other elements described in the instant specification. Unless otherwise explicitly stated, the use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or adapted by the skilled artisan to other designs without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

Additional features and functionality of the invention are described in the claims appended hereto. Such claims are hereby incorporated in their entirety by reference thereto in this specification and should be considered as part of the application as filed.

Multiple variations and modifications are possible in the embodiments of the invention described here. Although certain illustrative embodiments of the invention have been shown and described here, a wide range of changes, modifications, and substitutions is contemplated in the foregoing disclosure. While the above description contains many specific details, these should not be construed as limitations on the scope of the invention, but rather exemplify one or another preferred embodiment thereof. In some instances, some features of the present invention may be employed without a corresponding use of the other features. Accordingly, it is appropriate that the foregoing description be construed broadly and understood as being illustrative only, the spirit and scope of the invention being limited only by the claims which ultimately issue in this application.

1. A fluid sampling device including:
(a) body fluid sampling means adapted for applying against the skin of a test subject, optionally the body fluid sampling means adapted for self-sampling, the body fluid sampling means comprising a cutting device for making a laceration for fluid sampling of the test subject;
(b) optionally, analysis means configured for using one or more droplet(s) of the sampled fluid to analyze the fluid; and
(c) a medical analysis vacuum tube adapted to be filled with a sample of fluid for analysis in a point of care or medical lab, and a needle for interfacing with the medical analysis vacuum tube meeting size and interface standards, the vacuum tube adapted to be filled with a sample of fluid for analysis at a point of care or medical lab and to provide suction necessary to fill the vacuum tube with the fluid.

2. A method of fluid sampling using the device of claim 1, in a first step, a test subject is tested for a pathogen, and, if tested positive, in a second step, a treatment and/or quarantine protocol is initiated to ensure that a test subject having a positive test is handled in a manner which helps minimize the spread of the pathogen.

3. A lancet adapted for making a laceration in the skin of a user/patient for a fluid sampling device of claim 1 used for collecting a body fluid, wherein the cutting device is the lancet and the lancet is made in one piece of material and is disposed in a holder so as to enable its providing of the energy for movement and the guiding of its movement.

4. The sampling device of claim 1, wherein a fluid extraction passage or conduit from the patient's wound to the vacuum tube is formed in a needle.

5. (canceled)

6. (canceled)

7. The fluid sampling device of claim 1, wherein the fluid is, at least in part, blood.

8. The fluid sampling device of claim 1, wherein the fluid is, at least in part, puss.

9. The fluid sampling device of claim 1, wherein the fluid is, at least in part, venom.

10. The fluid sampling device of claim 1, wherein the device is adapted to use one or more droplet(s) of the sampled body fluid, to analyze the body fluid.

11. (canceled)

12. The fluid sampling device of claim 1, wherein the medical analysis tube is a standard medical analysis tube.

13. A method of capillary blood sampling, wherein the method is adapted for self sampling, the method includes at least the steps of:
a) placing an adhesive pad at the intended location for the capillary blood sampling, the adhesive pad including at least one mechanical localization feature and optionally visual indications;
b) lacerating the user/patient's skin with the cutting device of the sampling device of claim 1, thereby opening at least one wound in the user/patient's skin, the cutting device being positioned using the at least one mechanical localization feature of the adhesive pad;
c) collecting the patient's capillary blood with a fluid sampling device, the device being positioned on the at least one wound using the at least one mechanical localization feature of the adhesive pad and the device using vacuum to collect blood from the at least one wound and to fill a sample analysis tube; and
d) dressing the at least one wound using a foldable part of the adhesive pad.

14. The fluid sampling device of claim 1, including an adhesive pad, the adhesive pad adapted for application at the intended location for the capillary blood sampling, the adhesive pad including at least one mechanical localization feature and optionally visual indications, the adhesive pad further containing at least one mechanical feature that allows for the precise placement of the cutting device and of the fluid sampling device and a foldable portion adapted for covering the at least one wound after the blood collection.

15. The fluid sampling device of claim 1, or wherein the cutting device includes a lacerator including a lancet-adapted for making a one or more lacerations in the skin of a user/patient, wherein the lancet construction is made in one piece of material and provides the energy and the guiding for its movement.

16. The device of claim 1, wherein the cutting device includes a lacerator including a lancet adapted for making a one or more lacerations in the skin of a user/patient, wherein the lacerator contains one blade for each of the one or more lacerations and makes the one or more lacerations simultaneously.

17. The lacerator of claim 14, wherein when lacerating the patient's skin the trajectory of the at least one blades follows a trajectory that combines at least one rotary and at least one linear component of movement.

18. (canceled)

19. A lancet adapted for making a laceration in and under the surface of the skin of a user/patient for the sampling device of claim 1 used for collecting a body fluid, wherein the cutting device follows a trajectory that creates a laceration under the surface of the user/patient's skin which is longer than the laceration it makes at the surface of the user/patient's skin, thereby favoring a quick healing of the wound after body fluid collection.

20. The fluid sampling device of claim 4, wherein the medical analysis tube is a standard medical analysis tube.

21. The fluid sampling device of claim 5, wherein the medical analysis tube is a standard medical analysis tube.

22. The fluid sampling device of claim 6, wherein the medical analysis tube is a standard medical analysis tube.

23. The fluid sampling device of claim 7, wherein the medical analysis tube is a standard medical analysis tube.

24. The fluid sampling device of claim 8, wherein the medical analysis tube is a standard medical analysis tube.
